(19) United States
(12) Patent Application Publication
(10) Pub. No.: US 2025/0375621 A1
(43) Pub. Date: Dec. 11, 2025

(54) PHOTODYNAMIC THERAPY DEVICE AND METHOD

(71) Applicant: University of Rochester, Rochester, NY (US)

(72) Inventors: Timothy M. Baran, Rochester, NY (US); Nicole A. Wilson, Rochester, NY (US); Korry Wirth, Rochester, NY (US); Phuong Le, Rochester, NY (US); Mitchell Kershner, Rochester, NY (US)

(73) Assignee: University of Rochester, Rochester, NY (US)

(21) Appl. No.: 19/226,941

(22) Filed: Jun. 3, 2025

Related U.S. Application Data
(60) Provisional application No. 63/656,780, filed on Jun. 6, 2024.

(51) Int. Cl. A61N 5/06

(52) U.S. Cl. CPC A61N 5/062; A61N 5/0601; A61N 5/0624; A61N 2005/0612; A61N 2005/063; A61N 2005/0663

(57) ABSTRACT

Aspects of the present invention relate to a photodynamic therapy (PDT) device having a cannula having proximal and distal ends and a lumen therebetween, a flexible tube having proximal and distal ends, wherein the proximal end is attached to and extending out from the distal end of the cannula, a handle portion attached to the proximal end of the cannula including a housing with an adjustment mechanism, wherein adjusting the adjustment mechanism articulates the flexible tube, one or more conduits extending from at least the handle portion through the cannula to the distal end of the flexible tube, and a light source positioned at the distal end of the flexible tube, configured to irradiate in a distal direction. Further, a PDT method is disclosed.

### CROSS-REFERENCE TO RELATED APPLICATIONS

This application claims priority to U.S. Provisional Patent Application No. 63/656,780, filed Jun. 6, 2024, incorporated herein by reference in its entirety.

### STATEMENT REGARDING FEDERALLY SPONSORED RESEARCH OR DEVELOPMENT

This invention was made with government support under AI178152 awarded by the National Institutes of Health. The government has certain rights in the invention.

### BACKGROUND OF THE INVENTION

Appendicitis is the most common general surgical condition affecting children. The condition contributes to more morbidity and health care resource utilization than any other pediatric general surgical condition. Approximately ˜30% of pediatric cases are considered complicated (or perforated) appendicitis. In addition, this specific subpopulation suffers from the majority of the morbidity. Of this subpopulation, 13% require drain placement after appendectomy, 11% return to the emergency department (ED) within 30 days, 34.5% require postoperative imaging, and patients with complicated or perforated appendicitis have an overall a mean length of stay of 6.48 (3.6) days. In perforated appendicitis and other acute abdominal maladies, widespread infection can develop throughout the peritoneal cavity. This is typically treated with intravenous antibiotics, which results in extended hospital stays to allow time for antibiotics to control intra-abdominal infection due to stool and bacteria leakage. Shortening or eliminating the hospital stay could drastically improve patient experience and reduce hospital costs.

Photodynamic Therapy (PDT) is a promising treatment modality for oncology and antimicrobial applications that relies on the excitation of light-sensitive drugs known as photosensitizers by visible light in order to generate reactive oxygen species (ROS). As the antimicrobial effects of PDT occur on the timescale of hours, rather than the days required for traditional antibiotics, PDT can significantly reduce hospital stays for these patients. While PDT has been used to treat other infections, it has never been applied to intra-abdominal infection.

Thus, there is the need in the art for PDT devices and methods for treating patients with intraabdominal contamination. The present invention meets this need.

### SUMMARY OF THE INVENTION

A photodynamic therapy (PDT) device comprises a cannula having proximal and distal ends and a lumen therebetween, a flexible tube having proximal and distal ends, wherein the proximal end is attached to and extending out from the distal end of the cannula, a handle portion attached to the proximal end of the cannula comprising a housing with an adjustment mechanism, wherein adjusting the adjustment mechanism articulates the flexible tube, one or more conduits extending from at least the handle portion through the cannula to the distal end of the flexible tube, and a light source positioned at the distal end of the flexible tube, configured to irradiate in a distal direction.

In some embodiments, the device further comprises first and second guidewires positioned inside first and second conduits of the one or more conduits, each guide wire connected to the adjustment mechanism and the flexible tube. In some embodiments, the light source comprises a fiberoptic cable extending through a third conduit of the one or more conduits, wherein the third conduit extends out of the handle portion proximally.

In some embodiments, the adjustment mechanism comprises an adjustment lever and ratcheting mechanism, each at least partially housed inside the handle portion, wherein the ratcheting mechanism is connected to the first and second guidewires and rotated by the adjustment lever. In some embodiments, the adjustment mechanism comprises a locking lever extending out from the handle portion connected to the ratcheting mechanism, and wherein the adjustment mechanism articulates the flexible tube along at least one axis, and the locking lever locks the position of the adjustment mechanism. In some embodiments, the adjustment mechanism articulates the flexible tube within a range, wherein the range is between 0°-270°.

In some embodiments, the device further comprises a fourth conduit of the one or more conduits configured to dispense one or more fluids, wherein the one or more fluids comprise one or more agents selected from the group consisting of: therapeutic agent, photosensitizing agent, photosensitive therapeutic agent, antimicrobial agent, antimicrobial photosensitive therapeutic agent, and antibiotic agent. In some embodiments, the photosensitizing agent comprises at least one selected from the group consisting of: methylene blue, indocyanine green, and 5-Aminolevulinic acid. In some embodiments, the one or more fluids further comprise a light scattering emulsion selected from: lipid emulsion, sterile lipid emulsion, intravenous fat emulsion, intralipid, and nutrilipid.

In some embodiments, the device further comprises a fifth conduit of the one or more conduits comprising one or more sensors, wherein the one or more sensors are selected from the group consisting of: detector, photodiode, thermistor, transducer, photodiode configured to detect fluorescence, spectrometer, and fiber-coupled spectrometer. In some embodiments, the device further comprises a camera positioned at the distal end of the flexible tube.

In some embodiments, the device further comprises a sixth conduit of the one or more conduits configured to produce suction to remove fluid dispensed from the fourth conduit, or to retrieve a biological sample, wherein the biological sample comprises any of: fluid, biological fluid, purulent fluid, tissue, excised tissue, microbe, and microbial community.

In some embodiments, the light source is configured to emit one or more wavelengths of light ranging between 500 nm-900 nm, between 600 nm-850 nm, between 625 nm-785 nm, between 615 nm-645 nm, between 650 nm-680 nm, between 770 nm and 800 nm, or optionally configured to emit one or more target wavelengths, wherein the wavelengths are selected from: 633 nm, 665 nm, and 785 nm.

A method for performing PDT comprises the steps of providing any disclosed PDT device, delivering one or more fluids to an area of interest of a subject, positioning the distal end of the flexible tube near the area of interest, adjusting the distal end of the flexible tube to the area of interest, and irradiating at least a portion of the area of interest with the light source.

In some embodiments, the one or more fluids are delivered to the area of interest through the one or more conduits, and wherein the one or more fluids comprise one or more agents selected from the group consisting of: therapeutic agent, photosensitizing agent, photosensitive therapeutic agent, antimicrobial agent, antimicrobial photosensitive therapeutic agent, and antibiotic agent. In some embodiments, the photosensitizing agent comprises at least one selected from the group consisting of: methylene blue, indocyanine green, and 5-Aminolevulinic acid. In some embodiments, the one or more fluids further comprise a light scattering emulsion selected from: lipid emulsion, sterile lipid emulsion, intravenous fat emulsion, intralipid, and nutrilipid.

In some embodiments, the light source is configured to emit one or more wavelengths of light ranging between 500 nm-900 nm, between 600 nm-850 nm, between 625 nm-785 nm, between 615 nm-645 nm, between 650 nm-680 nm, between 770 nm and 800 nm, or optionally configured to emit one or more target wavelengths, wherein the wavelengths are selected from: 633 nm, 665 nm, and 785 nm.

In some embodiments, the method further comprises the step of removing at least a portion of the one or more fluids through the one or more conduits. In some embodiments, the method further comprises the step of retrieving a biological sample from the area of interest of the subject, wherein the biological sample is retrieved through the one or more conduits.

In some embodiments, the method further comprises the step of imaging the area of interest with a camera positioned at the distal end of the flexible tube. In some embodiments, the method further comprises the step of measuring fluorescence at the area of interest with a sensor positioned at the distal end of the flexible tube. In some embodiments, the area of interest of the subject is selected from the group consisting of: bodily cavity, abdominal cavity, peritoneum, chest cavity, gastrointestinal tract, thoracic cavity, spinal cavity, pelvic cavity, abdominopelvic cavity, inside the bladder, inside the tracheobronchial tree, spinal canal, inside the GI tract, inside the mouth, inside the pharynx.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A is an image depicting a side view of an exemplary photodynamic therapy (PDT) device comprising a proximal handle portion, a cannula, and a distal flexible tube according to aspects of the present invention.

FIG. 1B is an image depicting an enlarged side view of an exemplary cannula and flexible tube for an exemplary PDT device with the flexible tube articulated at least 90° according to aspects of the present invention.

FIG. 1C is an image depicting a front perspective view of an exemplary flexible tube.

FIG. 1D is an image depicting a see-through perspective view of the flexible tube of FIG. 1C.

FIG. 1E is an image depicting a rear perspective view of the flexible tube of FIG. 1C.

FIG. 1F is an image depicting a cutaway front perspective view of an exemplary handle portion with adjustment mechanism.

FIG. 1G is an image depicting a side view of an exemplary handle portion and adjustment mechanism.

FIG. 1H is an image depicting a side view of a prototype for an exemplary PDT device.

FIG. 1I is an image depicting a side cutaway view of an exemplary handle portion and adjustment mechanism for a PDT device prototype.

FIG. 1J is an image depicting a side view of an exemplary PDT device according to aspects of the present invention.

FIG. 1K is an image depicting a side view of a portion of an exemplary flexible tube of a PDT device.

FIG. 1L is an image depicting a cross-sectional view of an exemplary flexible tube of a PDT device.

FIG. 2 is a diagram depicting an exemplary architecture for a computer.

FIG. 3 is a diagram depicting an exemplary PDT method according to aspects of the present invention.

FIGS. 4A-4D are plots showing a reduction of colony forming units (CFUs) in four representative bacteria species following in vitro PDT using the disclosed PDT device and method. FIG. 4A is a plot showing CFU/ml of MRSA after PDT treatment, with and without Methylene Blue (MB). FIG. 4B is a plot showing CFU/ml of E. faecalis after PDT treatment, with and without MB. FIG. 4C is a plot showing CFU/ml of E. coli K1+after PDT treatment, with and without MB. FIG. 4D is a plot showing CFU/ml of K. pneumoniae ckp1 after PDT treatment, with and without MB.

FIG. 5A is a diagram depicting an exemplary PDT method according to aspects of the present invention.

FIG. 5B is a diagram depicting an exemplary experimental setup for testing the disclosed PDT method.

FIG. 5C is a diagram (left) showing the biological fluid sampling statistics and the clinical microbiology of the samples, and a plot (right) showing the percent speciation of the samples.

FIG. 6A is a diagram depicting an enlarged view of the exemplary experimental setup of FIG. 5B showing a light source irradiating a well plate.

FIG. 6B is a plot showing the results of the experiment of FIG. 5B showing a reduction of E. coli.

FIG. 6C is a plot showing the results of the experiment of FIG. 5B showing a reduction of P. aeruginosa.

FIG. 6D is a plot showing the results of the experiment of FIG. 5B showing a reduction of S. anginosus.

FIG. 7 is a diagram depicting an exemplary perforated appendicitis model utilizing the disclosed PDT method.

FIGS. 8A-8J are a series of images showing the steps of the perforated appendicitis model of FIG. 7, as performed on a subject. FIG. 8A is an image showing an appendix on day 1 before an appendectomy. FIG. 8B is an image showing the appendectomy on day 2. FIG. 8C is an image showing an appendectomy on day 2. FIG. 8D is an image showing the administration of MB on day 2. FIG. 8E is an image showing post-MB administration on day 2. FIG. 8F is an image showing the optical fiber of the disclosed PDT device in intralipid solution on day 2. FIG. 8G is an image showing illumination of the optical fiber on day 2. FIG. 8H is an image showing post-MB PDT of the appendectomy. A control subject was also used that did not receive irradiation with a light source. FIG. 8I is an image showing the results of the control subject that was treated with only MB. FIG. 8J shows the results of the subject that received MB and was irradiated with a light source.

FIG. 9 is an image of a culture of fluid sampled from the subject shown in FIGS. 8A-8J, showing a reliable initiation of perforated appendicitis including clinical evidence of peritonitis and polymicrobial infection.

FIG. 10 is a plot showing the results for the pre- and post-fluid sampling of the subject shown in FIGS. 8A-8J.

### DETAILED DESCRIPTION

It is to be understood that the figures and descriptions of the present invention have been simplified to illustrate elements that are relevant for a clear understanding of the present invention, while eliminating, for the purpose of clarity many other elements found in related devices, systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” “user”, and the like are used interchangeably herein, and refer to any animal amenable to the systems, devices, and methods described herein. The patient, subject or individual may be a mammal, and in some instances, a rabbit, or a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention discloses various novel systems and methods for performing photodynamic therapy (PDT) on a subject. Although exemplary PDT devices are disclosed, it should be appreciated that aspects of the devices and methods disclosed herein may be operated in conjunction with any known and relevant laparoscopic system or device, surgical system or device, and/or surgical tool or instrument.

### Photodynamic Therapy Device

Disclosed herein is a photodynamic therapy (PDT) device for treating a subject with, in some examples, light and photosensitizers according to aspects of the present invention. In some embodiments, the disclosed device is a laparoscopic instrument comprising a handle portion with adjustment mechanism connected to a stiff laparoscopic cannula, a flexible articulating tube with a distal opening connected to the cannula and adjustment mechanism, and at least one light source positioned in the distal opening of the flexible tube. In some embodiments, a user is able to insert a fiber optic cable through the handle portion and advance the fiber through the cannula until it reaches the distal opening of the flexible tube. The user is then able to grasp the handle of the device with a single hand and control the flexible tube articulation with the adjustment mechanism. In some embodiments, the adjustment mechanism comprises an adjustment lever with locking lever, wherein squeezing the locking lever engages the adjustment lever to adjust the flexible tube. Once the device is positioned, the user may release the locking lever, and the adjustment mechanism holds the desired position. Then, in conjunction with one or more fluids or therapeutics, light from the light source is applied to treat the subject. In some embodiments, a fluid or therapeutic is dispensed through a conduit extending through the cannula to the flexible tube. In some embodiments, fluid and/or tissue at the area of interest may be sampled through separate conduits. In some embodiments, the disclosed PDT device comprises one or more sensors configured to capture and/or measure various metrics. In some embodiments, use of the disclosed PDT device produces reactive oxygen species (ROS) in an area of interest of a subject. In some embodiments, the disclosed PDT device and method produces antimicrobial effects or treatments, and/or disinfecting effects or treatments within a cavity of a subject, such as within the abdomen.

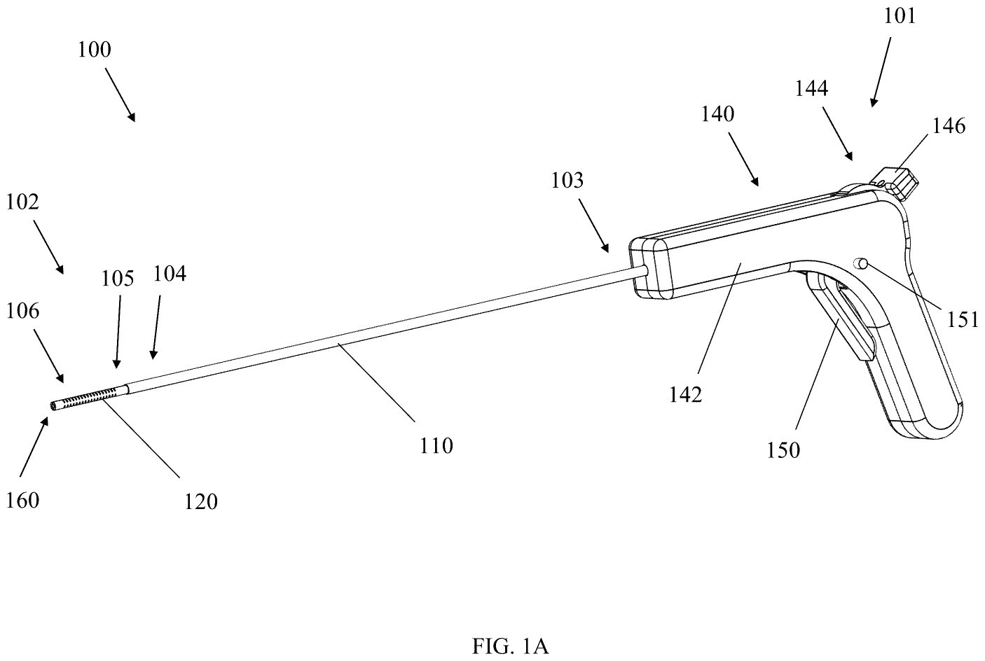

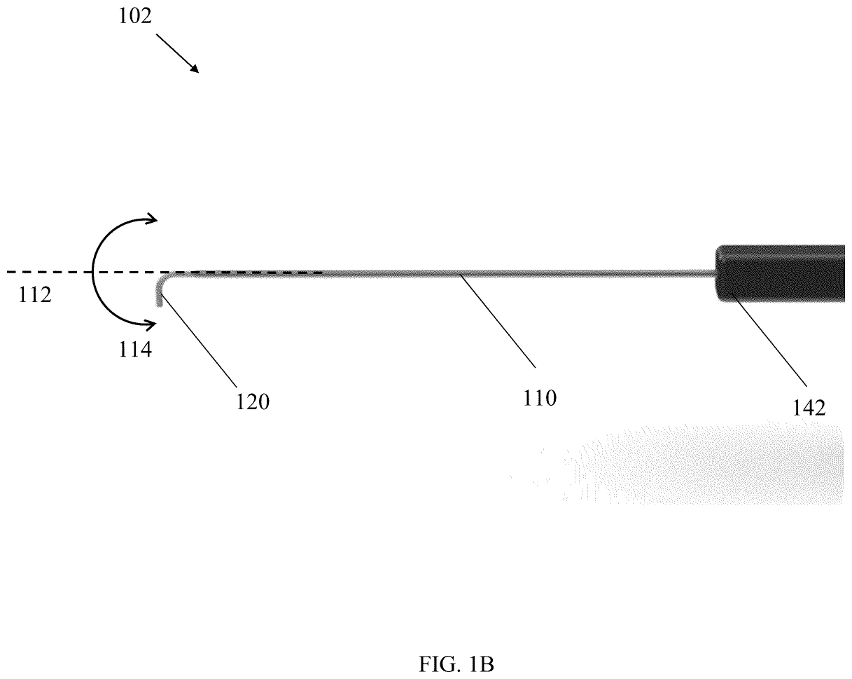

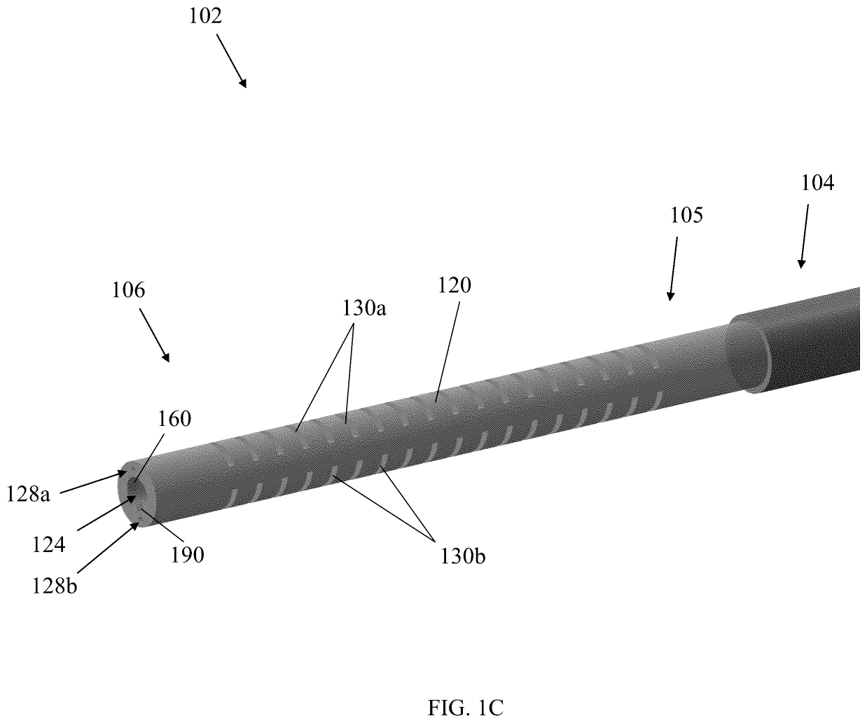

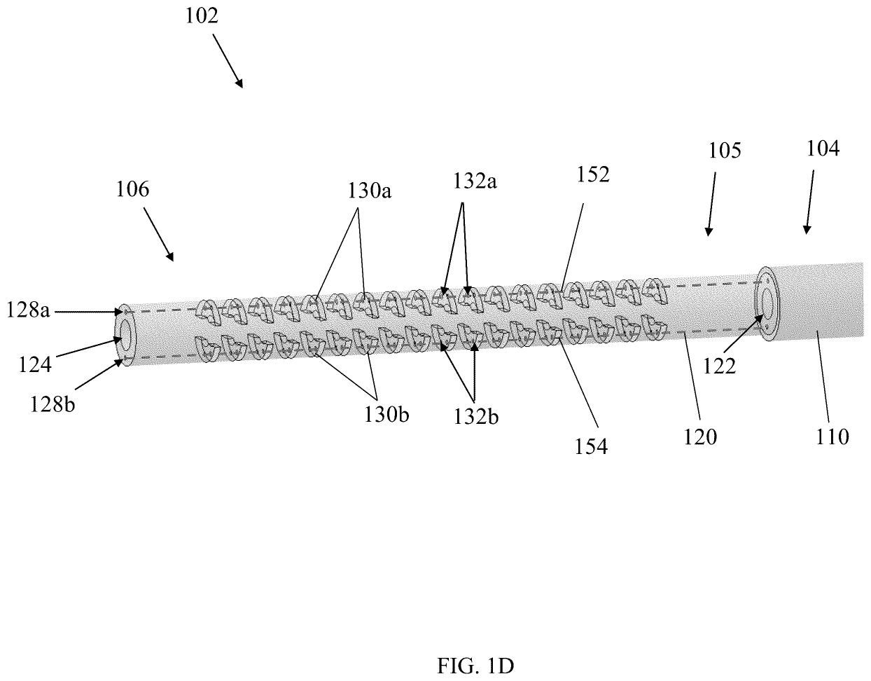

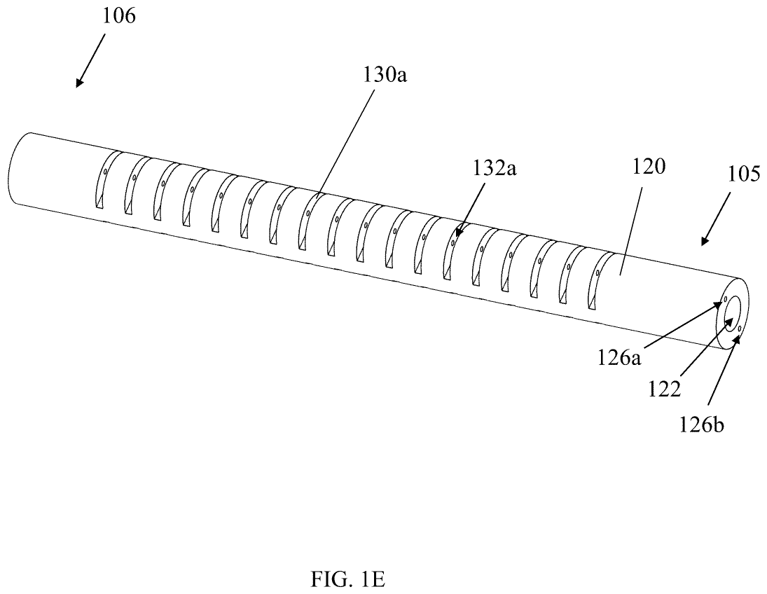

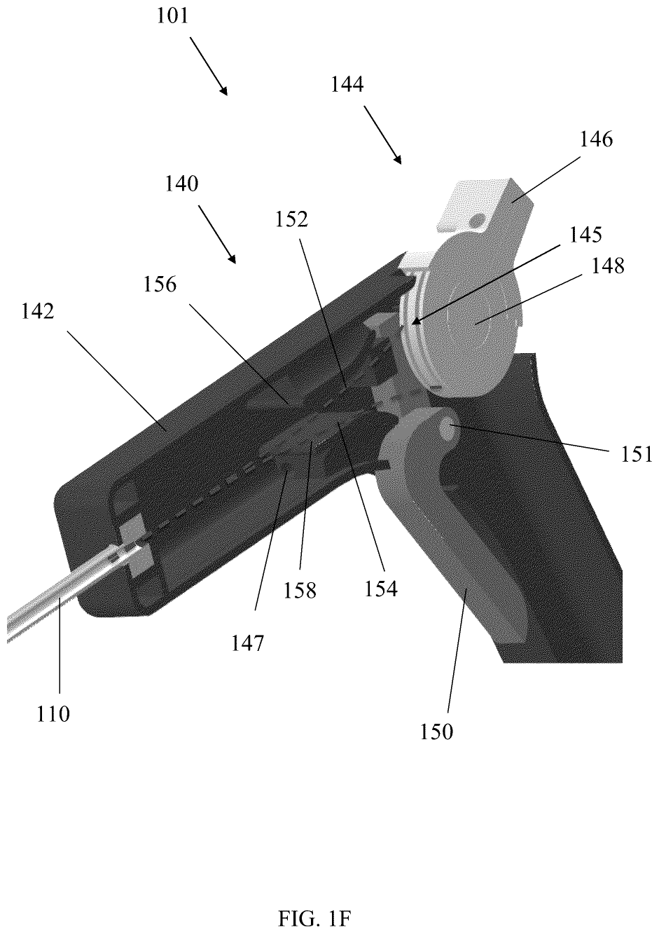

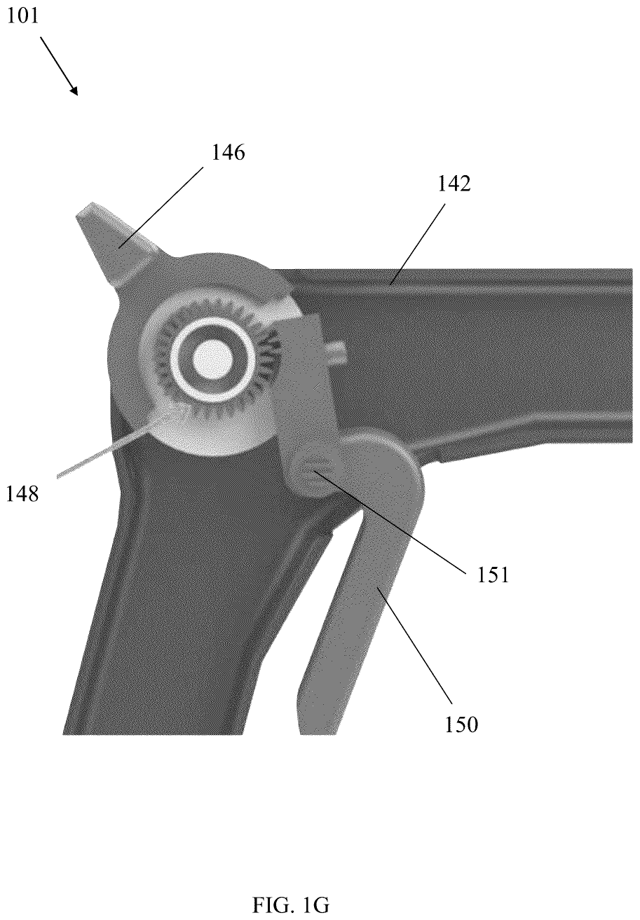

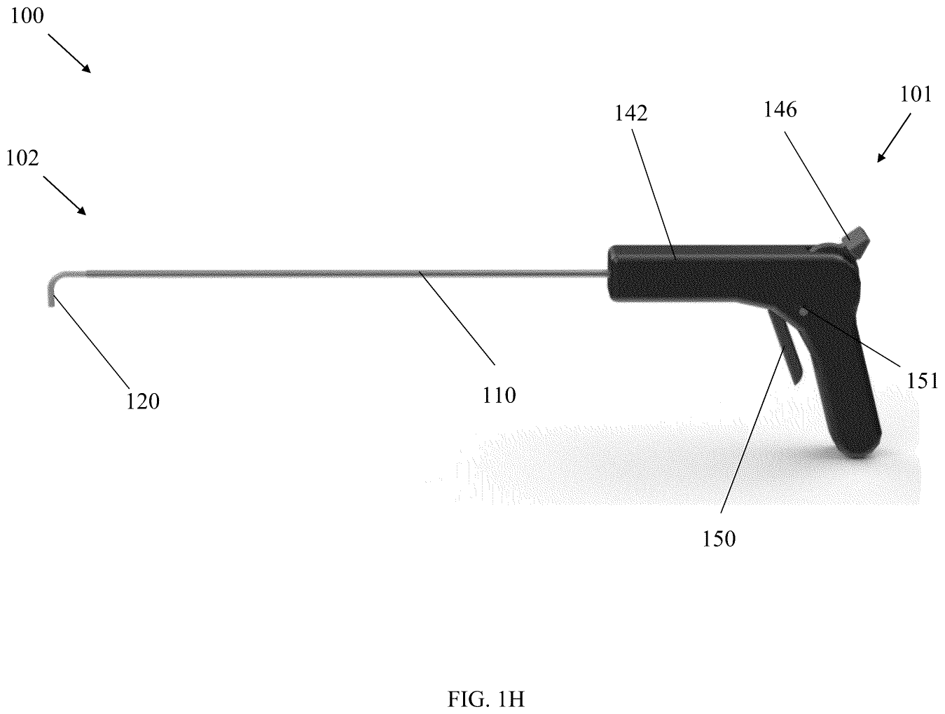

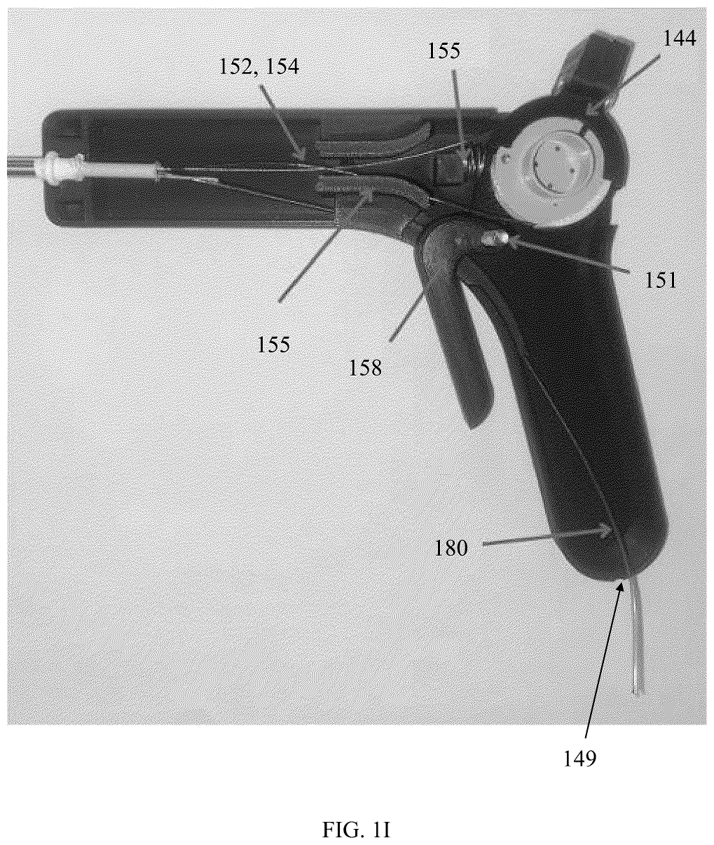

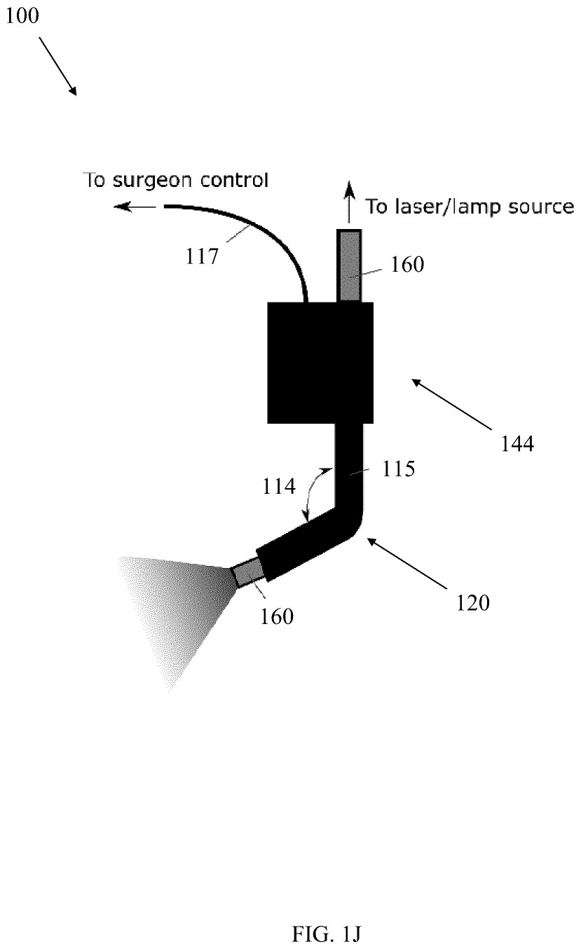

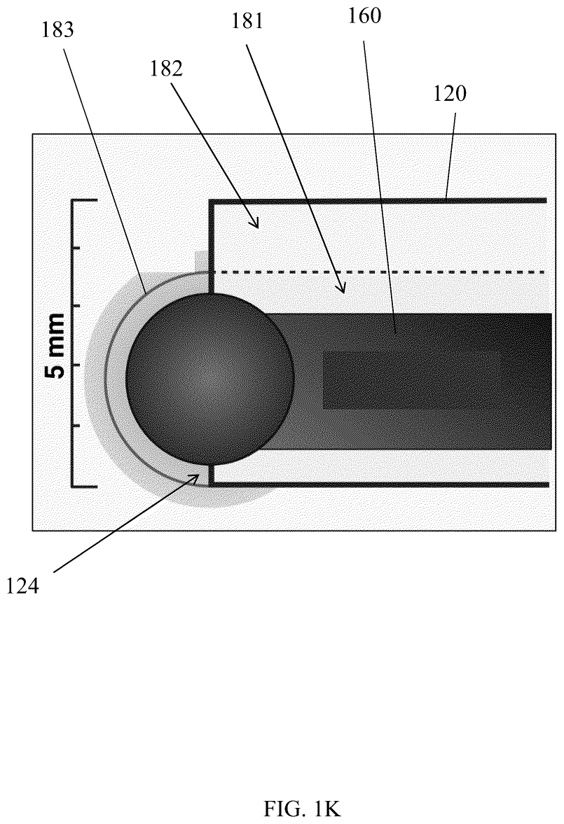

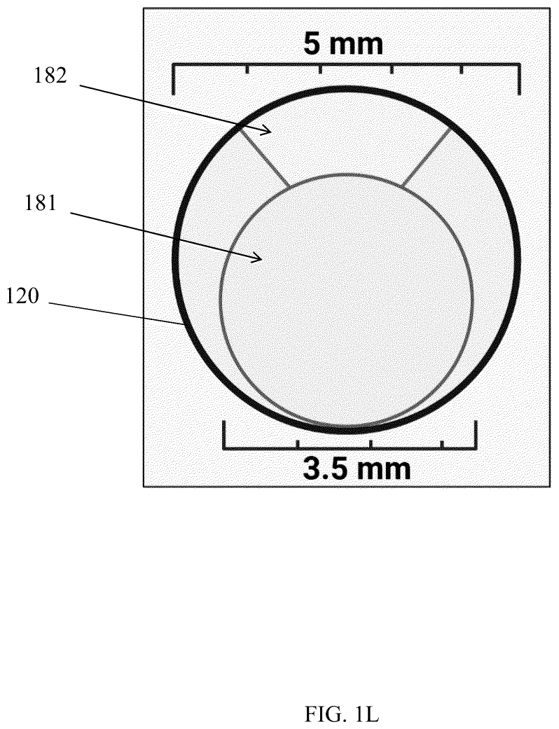

Aspects of the present invention relate to a PDT device for treating a subject. Referring now to FIG. 1A, shown is an exemplary PDT device 100 with a proximal end 101 comprising a handle portion 140, attached to a distal end 102 comprising a cannula 110 and flexible tube 120. Device 100 is configured to at least partially house or position a light source 160 at distal end 102. Cannula 110 has a proximal end 103 and a distal end 104 and is attached to flexible tube 120 having a proximal end 105 and distal end 106. In some embodiments, device 100 comprises a means of adjusting flexible tube 120. In some embodiments, handle portion 140 comprises a housing 142 with an adjustment mechanism 144, wherein adjusting adjustment mechanism 144 articulates and positions flexible tube 120. The at least one light source 160 is configured to illuminate in a range of directions based on the articulation or direction of flexible tube 120. In some embodiments, device 100 comprises one or more conduits 180 extending at least partially through the device for delivering a light source, sensor and/or fluid, or capturing a sample. In some embodiments, device 100 comprises one or more sensors 190 positioned at distal end 102.

Referring now to FIG. 1B, shown is an enlarged view of distal end 102 of an exemplary device 100 displaying an exemplary articulation range of flexible tube 120. Flexible tube 120 is configured to articulate or adjust about at least one axis and have an operational range or range of flexion. In some embodiments, adjustment mechanism 144 comprises a range of flexion that allows the user to adjust flexible tube 120 to an infinite number of positions about at least one axis 112, shown as range 114. In some embodiments, range 114 of flexible tube 120 ranges between 0° and 180°, or between 0° and 270°. In some embodiments, range 114 is about 180°, 190°, 200°, 210°, 220°, 230°, 240°, 250°, 260°, 270°, 280°, 290°, or about 300°.

Referring now to FIGS. 1C-1E, shown are various views of an exemplary flexible tube 120 according to aspects of the present invention. Flexible tube 120 generally comprises a proximal end 105 and distal end 106, with a proximal opening 122 and distal opening 124 forming a lumen therebetween. Flexible tube 120 allows for distal end 106 (and as a result distal end 102 of device 100) to articulate in order to position the at least one light source 160, one or more conduits 180, and/or one or more sensors 190 near, or directed at, one or more areas of interest of a subject. In some embodiments, flexible tube 120 is configured to receive one or more guidewires to pull on or push (or give slack) to a portion of flexible tube 120 to articulate distal end 106 to a desired position or angle. In some embodiments, adjustment mechanism 144 of handle portion 140 can be used to adjust the guidewires, thereby adjusting flexible tube 120 to a desired position or angle.

In some embodiments, flexible tube 120 comprises proximal guide wire openings 126a and 126b forming guide wire pathways through flexible tube 120 to distal guide wire openings 128a and 128b. In some embodiments, flexible tube 120 comprises lateral slots or recesses 130a and 130b, formed by cutout regions in the tube that allow easier flexing. In some embodiments, a series of guidewire pathway openings 132a and 132b are arranged in recesses 130a and 130b, connecting the guide wire pathways formed between proximal guidewire openings 126a and 126b, and distal guidewire openings 128a and 128b.

In some embodiments, recesses 130a and 130b comprise cutout regions formed in any size or shape. In some embodiments, there are any number of recesses 130a and 130b in flexible tube 120. For example, in some embodiments, recesses 130a and 130b have a number of recesses ranging between 1 and 50, or between 1 and 25, or between 10 and 20. In some embodiments, recesses 130a and 130b form cutout regions or slots that extends into flexible tube 120 about 25% of the diameter of flexible tube 120. However, the recesses 130a and 130b may extend into flexible tube 120 any percentage relative to the diameter of flexible tube 120. In certain embodiments, recesses 130a and 130b are approximately 1 mm wide. In certain embodiments, the recesses of recesses 130a are separated by a distance of about 4 mm. In certain embodiments the recesses of recesses 130b are separated by a distance of about 4 mm.

In some embodiments, cannula 110 and/or flexible tube 120 may comprise a sheath or sleeve surrounding at least a portion of the cannula or tube. For example, the sheath may comprise a flexible waterproof material covering at least a portion of flexible tube 120 and/or cannula 110 while leaving open or uncovered at least the distal opening 124. An exemplary sleeve 115 is shown in FIG. 1J.

Referring now to FIG. 1F, shown is an exemplary handle portion 140 comprising an adjustment mechanism 144 for adjusting the angle, position or articulation of flexible tube 120. Adjustment mechanism 144 controls a pair of guidewires that provide an adjustment means for the distal end 106 of flexible tube 120. Adjustment mechanism 144 comprises a guidewire retaining portion 145 configured to retain (e.g., wind and/or store) the guidewires. In some embodiments, the guidewires are wound in opposite directions within guidewire retaining portion 145 in order to adjust flexible tube 120. In some embodiments, adjustment mechanism 144 comprises an adjustment lever 146, a ratcheting mechanism 148, and a locking lever 150, at least some of which contained at least partially within housing 142. In some embodiments, adjustment lever 146 and locking lever 150 extend out at least a portion from housing 142 such that when handle portion 140 is gripped by a user, adjustment lever 146 is controlled by the user's thumb, and locking lever 150 is controlled by the remaining fingers. In some embodiments, ratcheting mechanism 148 is releasably connected to retaining portion 145 with a geared interface. In some embodiments, adjustment lever 146 is configured to rotate ratcheting mechanism 148 in order to position adjustment mechanism 144. In some embodiments, positioning adjustment mechanism 144 (e.g., adjustment lever 146 and/or ratcheting mechanism 148) adjusts or articulates flexible tube 120 via the one or more guidewires pushing or pulling on the distal end 106 of flexible tube 120.

In some embodiments, locking lever 150 is attached to housing 142 with a locking lever pin 151, such that locking lever 150 may pivot on an axis to lock and unlock the lever between at least two positions (e.g., a first locked position, and a second unlocked position). In some embodiments, locking lever 150 is configured to lock and unlock adjustment mechanism 144. In some embodiments, locking lever 150 may act as a clutch for adjustment mechanism 144 to apply variable force to the mechanism. For example, fully unlocking locking lever 150 may allow adjustment lever 146 to freely move, however only depressing the locking lever 150 half-way may add some resistance to the movement of adjustment lever 146. In some embodiments, adjustment mechanism 144 is unlocked when locking lever 150 is depressed or squeezed, and locked when locking lever 150 is released. In some embodiments, locking lever pin 151 is slidably attached to housing 142 and functions like a safety on a firearm, wherein the locking lever 150 may be enabled or disabled based on the position of locking lever pin 151. In some embodiments, ratcheting mechanism 148 further comprises a spring (not shown) positioned between the ratcheting mechanism 148 and housing 142, configured to return the ratcheting mechanism to a neutral or preset position. In some embodiments, when the ratcheting mechanism 148 is in the neutral position, flexible tube 120 extends out straight from cannula 110. In some embodiments, locking lever 146 further comprises a spring 155 (shown in FIG. 1I) positioned between a portion of locking lever 150 and housing 122, configured to bias locking lever 150 in a locked position, thereby also locking the position of flexible tube 120.

Aspects of the present invention relate to device 100 comprising one or more guidewires for positioning or adjusting flexible tube 160. Referring now to FIG. 1D and FIG. 1F, shown are exemplary guidewires extending from handle portion 140 to the distal end 106 of flexible tube 120. In some embodiments, device 100 comprises a first guidewire 152 and a second guidewire 154 extending through the lumen of cannula 110, each guide wire connected to the adjustment mechanism 144 and flexible tube 120. In some embodiments, guidewires 152 and 154 pass through dedicated conduits of the one or more conduits 180 (e.g., first and second conduits), in order to prevent friction as the cables move, or prevent abrasion of the cannula lumen and/or other conduits of the one or more conduits 180. In some embodiments, each of guidewire 152 and guidewire 154 attaches to positions near or inside distal guidewire openings 128a and 128b of flexible tube 120, respectively. In some embodiments, the distal ends of the guidewires close or cover the distal guide wire openings 128a and 128b. Guidewires 152 and 154 may be fixedly attached to flexible tube 120 using any attachment method, including, but not limited to, weldments, bolts, glues, adhesives, caps, end caps, or the like.

In some embodiments, handle portion 140 comprises one or more guidewire ramps positioned inside housing 142 configured to guide the guidewires to ratcheting mechanism 148. Referring again to FIG. 1F, in some embodiments, housing 142 comprises a first guidewire ramp 156, and a second guidewire ramp 158, positioned opposite guidewire ramp 156. In some embodiments, the guidewire ramps have smooth surfaces, or a low friction surfaces. In some embodiments, each guidewire ramp comprises sides or a retaining wall for guiding the guidewires. In some embodiments, each guidewire ramp comprises a curved surface for guiding the guidewires to the adjustment mechanism 144.

Aspects of the present invention relate to one or more conduits configured to house guidewires, light sources, or sensors, as well as enable fluid dispensing and fluid suction for device 100. Referring to FIG. 1I, in some embodiments, device 100 comprises one or more conduits 180 extending from proximal end 101 through cannula 110 to flexible tube 120. In some embodiments, the one or more conduits 180 comprise first and second conduits configured for guidewires extending between adjustment mechanism 144 and flexible tube 120. In some embodiments, the one or more conduits 180 comprise a third conduit (e.g., optical conduit 181 described herein) configured to provide illumination (e.g., by at least one light source 160). In some embodiments, a fourth conduit is configured for fluid dispensing (e.g., fluid conduit 182 described herein), a fifth conduit is configured for hosting one or more sensors 190, and a sixth conduit is configured for sampling (e.g., via suction, biopsy). In some embodiments, the light source 160 extends through the third conduit to a position at distal end 106 of flexible tube 120. In some embodiments, the one or more sensors 190 are positioned in flexible tube 120, or at distal end 106 of flexible tube 120.

In some embodiments, the one or more conduits 180 extend from the distal opening 124 of flexible tube 120 to the handle portion 140 of device 100. In some embodiments, the first and second conduits terminate within housing 142, with proximal openings directed towards ratcheting mechanism 148. In some embodiments, the one or more conduits 180 extend through a conduit channel 147 in housing 142 (shown in FIG. 1F). In some embodiments, the one or more conduits 180 extend out through handle portion 140 through one or more openings 149 in housing 142. In some embodiments, the fourth, fifth and sixth conduits extend out through the one or more openings 149 (shown in FIG. 1I). In some embodiments, the fourth conduit connects to any suitable fluid source (e.g., fluid pump with fluid reservoir).

Aspects of the present invention relate to using device 100 to deliver one or more fluids to a desired area of interest of a subject. In some embodiments, the one or more fluids is dispensed through at least one of the one or more conduits 180 (e.g., fluid conduit 182). In some embodiments, the one or more fluids comprises one or more agents selected from the group consisting of: therapeutic agent, photosensitizing agent, photosensitive therapeutic agent, antimicrobial agent, antimicrobial photosensitive therapeutic agent, and antibiotic agent. In one embodiment, the photosensitizing agent comprises at least one selected from the group consisting of methylene blue, indocyanine green, and 5-Aminolevulinic acid. In some embodiments, the one or more fluids comprises a light scattering emulsion selected from: lipid emulsion, sterile lipid emulsion, intravenous fat emulsion, intralipid, and nutrilipid.

Aspects of the present invention relate to at least one light source 160 directed outward from distal end 102 of device 100 configured to emit, irradiate or illuminate the one or more fluids with light at one or more wavelengths (i.e., activating a photosensitizer or photosensitizing agent with light). In some embodiments, light source 160 comprises a fiberoptic cable extending through the one or more conduits 180. Light source 160 may be connected to any suitable light emitting source known by one of ordinary level of skill in the art. In some embodiments, the light source 160 comprises a fiber optic cable optically connected to a medical laser, or medical lamp, configured to produce or emit relevant wavelengths of light. In some embodiments, light source 160 is configured to illuminate, emit or irradiate one or more wavelengths of light ranging between 500 nm-900 nm, between 600 nm-850 nm, or between 625 nm and 785 nm. In some embodiments, the one or more wavelengths of light range comprise wavelengths ranging between 615 nm-645 nm, between 650 nm-680 nm, or between 770 nm and 800 nm. In some embodiments, light source 160 is configured to emit one or more target wavelengths, wherein the wavelengths are selected from: 633 nm, 665 nm, and 785 nm. In some embodiments, the one or more wavelengths are chosen based upon the one or more photosensitizing agent dispensed or delivered to the area of interest. For example, but without limitation, 633 nm which corresponds to 5-Aminolevulinic acid, 665 nm which corresponds to methylene blue, and 785 nm which corresponds to indocyanine green.

Now referring to FIGS. 1K & 1L, depicted is an exemplary embodiment of flexible tube 120 of device 100. The flexible tube 120 may be at least partially housed within the cannula 110 of device 100. In some embodiments, the one or more conduits 180 comprise an optical conduit 181 and a fluid conduit 182 in flexible tube 120. The optical conduit 181 may be any conduit configured to provide illumination (e.g., by the at least one light source 160) described herein. The fluid conduit 182 may be any conduit configured for fluid dispensing described herein. It should be appreciated that the depicted embodiment of flexible tube 120 in FIGS. 1K & 1L may further comprise any number of additional conduits described herein.

Now referring to FIG. 1K in detail, depicted is a side view of a portion of an exemplary embodiment of flexible tube 120 of device 100. The depicted portion of flexible tube 120 comprises distal opening 124. Flexible tube 120 may comprise a shield 183 positioned over at least a portion of distal opening 124 and/or optical conduit 181. The shield 183 may be configured to form a barrier between at least some components of the flexible tube 120 and the exterior of the device 100. In some examples, the shield 183 may be configured to form a barrier between the optical conduit 181 including the light source 160 therein and the exterior of the device 100. In some embodiments, shield 183 is translucent such that light emitted from the light source 160 may reach the exterior of the device 100. In some embodiments, the shield 183 is optically clear or at least partially optically clear. In some embodiments, the shield 183 is translucent to certain light wavelengths or a certain range of light wavelengths.

In some embodiments, the shield 183 extends out a portion to the exterior of the device 100 from distal opening 124. The shield 183 may extend at any curvature. In some examples, the shield 183 is substantially dome-shaped, but also may be any concave shape, or flat. In some examples, the shield 183 may extend outward such that the end of a light source 160 may fit within the shield 183. The shield may comprise any materials known by one of ordinary skill in the art for use with a light source in a surgical device.

FIG. 1L depicts a cross-sectional view of an embodiment of the flexible tube 120 of device 100. The optical conduit 181 may be configured to house any light source 160, for example a fiber optic cable. As depicted, the diameter of the cross section of flexible tube 120 is or is about 5 mm, however it should be appreciated that the flexible tube 120 may have any dimensions appropriate for a desired use.

The cross section of optical conduit 181 may be substantially circular. The optical conduit 181 may have any dimensions. In some examples, the optical conduit 181 has a cross-sectional area or any other dimensions configured to retain a light source 160. In some embodiments, the optical conduit 181 has a diameter of or of about 3.5 mm. In some embodiments, at least a portion of the boundary of optical conduit 181 may comprise at least a portion of the boundary of flexible tube 120. In some embodiments, optical conduit 180 is offset within flexible tube 120.

The boundary of fluid conduit 182 may comprise at least a portion of the boundary of the optical conduit 181 and/or at least a portion of the boundary of flexible tube 120. For example, as depicted in FIG. 1K, the fluid conduit 182 may comprise a top boundary comprising flexible tube 120, a bottom boundary comprising optical conduit 181, and two side boundaries, each side boundary extending from the optical conduit 181 to the flexible tube 120. In some embodiments, fluid conduit 182 is irregularly shaped and offset from optical conduit 182. In some embodiments, fluid conduit 182 is polygonal shape, fan shaped, or wedge shaped. The optical conduit 181 and/or fluid conduit 182 as well as any additional conduits may extend to distal opening 124 of flexible tube 120.

In some embodiments, the sixth conduit (e.g., fluid conduit 182) connects to any suitable pump for creating suction and sampling biological fluids or tissue. In some embodiments, the sixth conduit is configured to remove fluid dispensed from the fourth conduit, or to retrieve a biological sample. In some embodiments, the biological sample comprises any of: fluid, biological fluid, purulent fluid, tissue, excised tissue, microbe, and microbial community.

In some embodiments, one or more sensors 190 communicatively and electronically connect to any suitable computing device (e.g., computer 200 described herein). In some embodiments, the one or more sensors 190 are selected from the group consisting of: detector, photodiode, thermistor, transducer, photodiode configured to detect fluorescence, spectrometer, and fiber-coupled spectrometer. In some embodiments, the one or more sensors 190 comprises a camera, or any sensor 265 of computer 200 described herein. In some embodiments, device 100 comprises display or screen (e.g., and LCD display) attached to or embedded within housing 142, electronically connected to the one or more sensors 190 and computing device. In some embodiments, the computing device (e.g., computer 200) is at least partially housed within housing 142. Aspects of the present invention relate to materials and dimensions for device 100. In some embodiments, device 100 is formed or manufactured from, or comprises one or more materials selected from: plastic, biocompatible plastic, vinyl, metal, synthetic, polymer, flexible material, rigid material, non-porous material, elastic material, sterilizable material, PLA, TPU. Flexible tube 120 may be formed from any suitable material that allows elastic flexing of the tube without deformation. Guidewires 152 and 154 may be formed from any suitably inelastic cable or wire known by one of ordinary level of skill in the art, including but not limited to, cable, wire, braided cable or wire, metal wire, metal allow wire, synthetic wire, or the like. In one example, handle portion 140 comprises one or more plastic materials, cannula 110 comprises one or more metal and/or plastic materials, and flexible tube 120 comprises any flexible material such as plastic, biocompatible plastic, metal, or combinations thereof. Any portion of device 100 (e.g., cannula 110, flexible tube 120, sheath) may comprise a one or more coatings, such as any biocompatible coating or biomaterial coating known by one of ordinary level of skill in the art.

Device 100 may be sized appropriately for the intended subject. Further, handle portion 140 should be sized to fit in a user's hand such that they may grip handle portion 140 and comfortably actuate adjustment mechanism 144. In some embodiments, device 100 has an overall length ranging between 10 cm and 100 cm. In some embodiments, flexible tube 120 has a length ranging between 1 cm and 10 cm, and a width or diameter ranging between 1 mm and 2 cm. In some embodiments, cannula 110 has a length ranging between 5 cm and 30 cm, between 10 cm and 25 cm, or between 18 cm and 25 cm. In some embodiments, cannula 110 has a width or diameter ranging between 1 mm and 3 cm, or between 1 mm and 1 cm, or has a width or diameter of less than 5 mm, or less than 4 mm. In some embodiments, the one or more guidewires have inner diameters ranging between 0.1 mm and 0.5 cm, and outer diameters ranging between 0.2 mm and 1 cm.

In some embodiments, device 100 is configured as an attachment or end-effector for robotic surgery applications. FIG. 1J shows an exemplary embodiment of device 100 configured for robotic surgery applications. It should be appreciated that in this embodiment, handle portion 140 is replaced with a self-contained and/or motorized adjustment mechanism 144 with an interface for attaching to any robotic surgery platform. A simplified version of device 100 comprises only cannula 110, flexible tube 120, adjustment mechanism 144, and the guidewires thereof, and any number of light sources and conduits described herein. In some embodiments, adjustment mechanism 144 electronically connects to any robotic surgery platform and/or computing device with a control wire 117.

### Computing Device

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

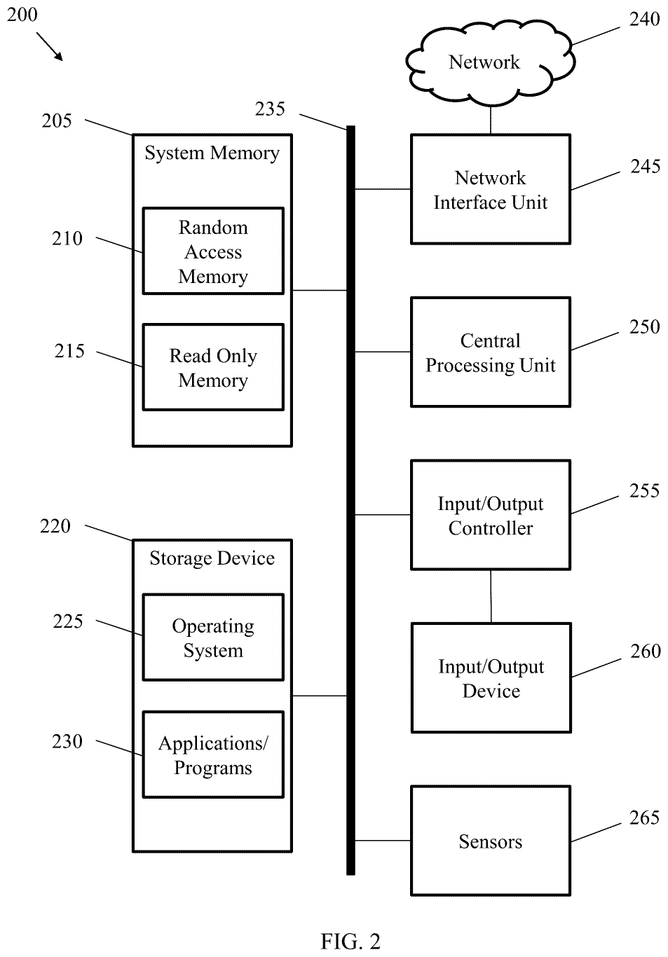

FIG. 2 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 2 depicts an illustrative computer architecture for a computer 200 for practicing the various embodiments of the invention. The computer architecture shown in FIG. 2 illustrates a conventional personal computer, including a central processing unit 250 (“CPU”), a system memory 205, including a random access memory 210 (“RAM”) and a read-only memory (“ROM”) 215, and a system bus 235 that couples the system memory 205 to the CPU 250. A basic input/output system containing the basic routines that help to transfer information between elements within the computer, such as during startup, is stored in the ROM 215. The computer 200 further includes a storage device 220 for storing an operating system 225, application/program 230, and data.

The storage device 220 is connected to the CPU 250 through a storage controller (not shown) connected to the bus 235. The storage device 220 and its associated computer-readable media provide non-volatile storage for the computer 200. Although the description of computer-readable media contained herein refers to a storage device, such as a hard disk or CD-ROM drive, it should be appreciated by those skilled in the art that computer-readable media can be any available media that can be accessed by the computer 200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 200 may operate in a networked environment using logical connections to remote computers through a network 240, such as TCP/IP network such as the Internet or an intranet. The computer 200 may connect to the network 240 through a network interface unit 245 connected to the bus 235. It should be appreciated that the network interface unit 245 may also be utilized to connect to other types of networks and remote computer systems.

The computer 200 may also include an input/output controller 255 for receiving and processing input from a number of input/output devices 260, including a keyboard, a mouse, a touchscreen, a camera, a microphone, a controller, a joystick, or other type of input device. Similarly, the input/output controller 255 may provide output to a display screen, a printer, a speaker, or other type of output device. The computer 200 can connect to the input/output device 260 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 220 and/or RAM 210 of the computer 200, including an operating system 225 suitable for controlling the operation of a networked computer. The storage device 220 and RAM 210 may also store one or more applications/programs 230. In particular, the storage device 220 and RAM 210 may store an application/program 230 for providing a variety of functionalities to a user. For instance, the application/program 230 may comprise many types of programs such as a word processing application, a spreadsheet application, a desktop publishing application, a database application, a gaming application, internet browsing application, electronic mail application, messaging application, and the like. According to an embodiment of the present invention, the application/program 230 comprises a multiple functionality software application for providing word processing functionality, slide presentation functionality, spreadsheet functionality, database functionality and the like.

The computer 200 in some embodiments can include a variety of sensors 265 (e.g., one or more sensors 190) for monitoring the environment surrounding and the environment internal to the computer 200. These sensors 265 can include a Global Positioning System (GPS) sensor, gyroscope, magnetometer, thermometer, proximity sensor, accelerometer, microphone, biometric sensor, barometer, humidity sensor, radiation sensor, detector, photodetector, photosensitive sensor or any other suitable sensor.

### PDT Method

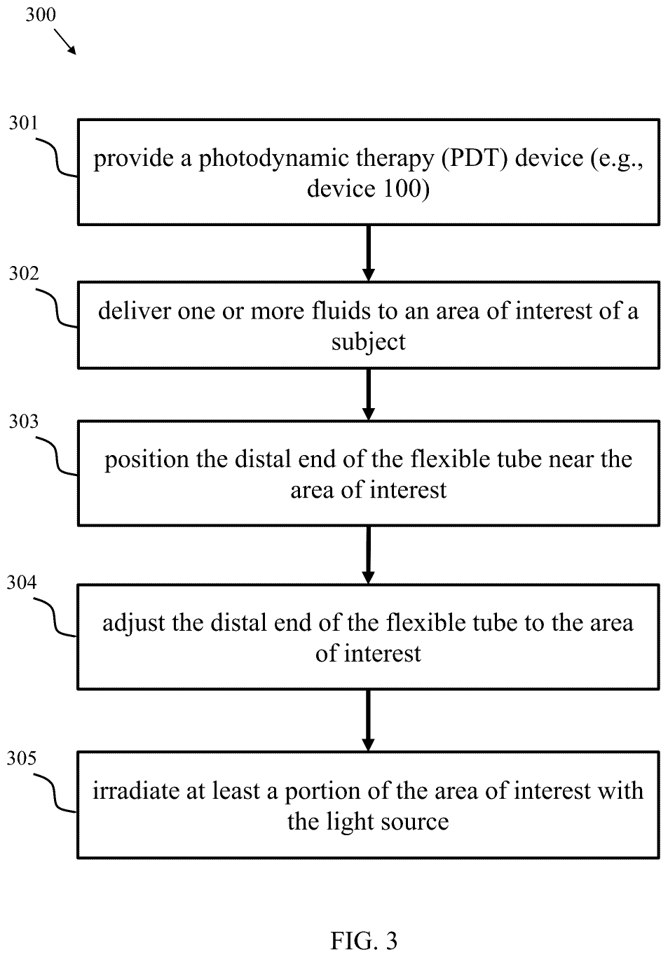

Aspects of the present invention relate to various PDT methods using a PDT device (e.g., device 100 disclosed herein). Referring now to FIG. 3, shown is an exemplary PDT method 300. In some embodiments, method 300 comprises the steps of 301 providing a PDT device (e.g., device 100); 302 delivering one or more fluids to an area of interest of a subject; 303 positioning the distal end of the flexible tube near the area of interest; 304 adjusting the distal end of the flexible tube to the area of interest; and 305 irradiating at least a portion of the area of interest with the light source. In some embodiments, the steps of positioning and/or adjusting the flexible tube may comprise positioning the PDT device (e.g., the cannula and/or flexible tube) near the area of interest, moving the PDT device in a distal or proximal direction, rotating the PDT device via the handle portion, and/or adjusting the adjustment mechanism to position the distal end of the flexible tube.

Aspects of the present invention relate to treating infections and/or infections as a result of a procedure, for example, but without limitation, treating an infection in a body cavity. In some embodiments, the disclosed PDT device or method may be used to treat: infections, appendicitis, acute abdominal maladies, intra-abdominal infection, infection in the peritoneal cavity, empyema (infection surrounding the lung), pneumonia, and endodontic infections. In some embodiments, the disclosed PDT device or method may be used to treat infections as a result of: surgical interventions or procedures, surgical excisions, incisions, wounds, onychomycosis, chromoblastomycosis, intra-abdominal abscess, appendectomy, diverticulitis, perforation of the GI tract or any other hollow viscus organ, and bronchitis.

Aspects of the present invention relate to delivering one or more fluids to an area of interest of a subject. In some embodiments, the one or more fluids is delivered to the area of interest through one or more conduits of the PDT device. In some embodiments, the one or more fluids comprises one or more agents selected from the group consisting of: therapeutic agent, photosensitizing agent, photosensitive therapeutic agent, antimicrobial agent, antimicrobial photosensitive therapeutic agent, and antibiotic agent. In one embodiment, the photosensitizing agent comprises at least one selected from the group consisting of methylene blue, indocyanine green, and 5-Aminolevulinic acid. In some embodiments, the one or more fluids comprises a light scattering emulsion selected from: lipid emulsion, sterile lipid emulsion, intravenous fat emulsion, intralipid, and nutrilipid. In some embodiments, the one or more fluids are delivered all at once, or in sequence, or with a delay. For example, in some embodiments, a photosensitizing agent is delivered to the area of interest with a light scattering emulsion. In some embodiments, the light scattering emulsion is delivered after the photosensitizing agent.

In some embodiments, the light source is configured to emit, irradiate, or illuminate one or more wavelengths of light ranging between 500 nm-900 nm, between 600 nm-850 nm, or between 625 nm-785 nm. In some embodiments, the one or more wavelengths of light range comprise wavelengths ranging between 615 nm-645 nm, between 650 nm-680 nm, or between 770 nm-800 nm. In some embodiments, the light source is configured to emit one or more targeted wavelengths, wherein the wavelengths are selected from: 633 nm, 665 nm, and 785 nm. In some embodiments, the one or more wavelengths are chosen based upon the one or more photosensitizing agent dispensed or delivered to the area of interest. For example, 633 nm which corresponds to 5-Aminolevulinic acid, 665 nm which corresponds to methylene blue, and 785 nm which corresponds to indocyanine green. In some embodiments, any disclosed PDT method further comprises the step of delivering 5-Aminolevulinic acid to an area of interest and irradiating the area of interest with 633 nm light. In some embodiments, any disclosed PDT method comprises the step of delivering methylene blue to an area of interest and irradiating the area of interest with 665 nm light. In some embodiments, any disclosed PDT method comprises the step of delivering indocyanine green to an area of interest and irradiating the area of interest with 665 nm light. It should be appreciated that disclosed fluid or agent may be used alone or in combination with other disclosed fluids or agents, and the area of interest may be suitably irradiated with the corresponding wavelengths of light.

In some embodiments, the any disclosed PDT method further comprises the step of removing a fluid, sample, or at least a portion of the therapeutic agent through one or more conduits of the therapy device. In some embodiments, any disclosed PDT method further comprises the step of retrieving a biological sample from the area of interest of the subject. In some embodiments, the biological sample is retrieved through one or more conduits of the therapy device. In some embodiments, the any disclosed PDT method further comprises the step of imaging the area of interest with the therapy device.

In some embodiments, any disclosed PDT method further comprises the step of measuring fluorescence at the area of interest with the therapy device. In some embodiments, the area of interest of a subject is selected from the group consisting of: body cavity, abdominal cavity, peritoneum, chest cavity, gastrointestinal tract, thoracic cavity, spinal cavity, pelvic cavity, abdominopelvic cavity, inside the bladder, inside the tracheobronchial tree, spinal canal, inside the GI tract, inside the mouth, inside the pharynx.

In some embodiments, power to the at light source is provided for at least one duration of time, wherein the duration of time ranges between 0.1 s and 45 min. In some embodiments, the duration of time is at least 10 s. In some embodiments, the at least one duration of time is 0.1 s, 0.2, s, 0.3 s, 0.4 s, 0.5 s, 0.6 s, 0.7 s, 0.8s, 0.9 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 33 s, 34 s, 35 s, 36 s, 37 s, 38 s, 39 s, 40 s, 41 s, 42 s, 43 s, 44 s, 45 s, 46 s, 47 s, 48 s, 49 s, 50 s, or any duration of time in between. In some embodiments, the duration of time is at least 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, or 30 min. In some embodiments the duration of time is about 1 min, about 2 min, about 5 min, about 10 min, about 15 min, about 20 min, about 25 min, or about 30 min. In some embodiments, the duration of time is dependent on the size of the area of interest, the tissue of interest, the structure of the tissue of interest, the subject, or the amount of therapeutic agent (e.g., methylene blue). In some embodiments, power to the light source is provided intermittently, or in a pattern.

In some embodiments, any disclosed PDT method further comprises the steps of measuring one or more metrics of the subject. In some embodiments, any disclosed PDT method comprises measuring fluorescence, reflectance, absorption, transmittance, or scattering, near or at the area of interest of the subject. In some embodiments, the measured fluorescence comprises emissions from endogenous fluorophores, or fluorescence emitted from administered photosensitizing agents or fluids. In some embodiments, fluorescence from endogenous fluorophores provides various metabolic information. In some embodiments, fluorescence, or one or more changes in fluorescence during treatment from an administered photosensitizing agent provides various information on ROS production. In some embodiments, measurement of broadband reflectance from tissue provides various information on tissue absorption and scattering properties. In some embodiments, these absorption properties allow for determination of tissue oxygenation status.

In some embodiments, the disclosed PDT device and method may be used as a combination therapy to augment efficacy and reduce side effects of existing drugs and therapies, such as with other antimicrobials, antibiotics, antifungals, surgeries or procedures, disease treatments or the like. In some embodiments, the disclosed PDT device and method may be used for drainage and/or lavage during any treatment, surgery or procedure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Photodynamic Therapy Device Prototype

FIG. 1H is an image depicting a side view of an exemplary photodynamic therapy (PDT) device prototype with the flexible tube articulated 90° according to aspects of the present invention. FIG. 1I is an image depicting a side cutaway view of an exemplary PDT device prototype.

FIG. 1H shows an exemplary PDT device prototype along with FIG. 1I which highlights the internal components located within the device handle. The disclosed prototype is a laparoscopic instrument generally comprising main parts such as a handle with integrated user interface components, a stiff laparoscopic shaft, an articulating tip, and central tubing to allow fiber optic delivery. The user is able to insert a fiber optic cable into the proximal end of the handle and advance the fiber through the central tubing until it reaches the distal articulating tip. The user is then able to grasp the handle of the device with a single hand and control any tip articulation with the wheel knob and the trigger components. Squeezing the trigger unlocks the ability to rotate the wheel knob to produce tip articulation in the up/down plane of FIG. 1H. Once proper articulation is set to position the fiber tip near the tissue of interest, the user may release the trigger, and the locking mechanism engages again to hold the desired tip articulation.

In some embodiments, the disclosed PDT device comprises the following components:

Articulating tip (also referred to herein as a flexible tube): A flexible plastic component on the most distal end of the device that is controlled by the articulating wheel knob (e.g., adjustment lever) and trigger mechanism (e.g., locking lever). The movement of the tip is due to two “drive wires” that extend through the upper and lower halves of the articulating tip and are attached to the articulating wheel.

Stiff Laparoscopic Shaft (also referred to herein as a cannula): A stainless-steel shaft that encases components that extend to the tip, such as the 2 drive wires and the central tubing. The diameter is <5 mm to allow interfacing with standard laparoscopic instrument ports, and the length measures 30 cm to match standard laparoscopic instruments.

Device Handle (also referred to herein as a handle portion): A plastic ergonomic handle that encases all of the internal device parts. The handle contains attachment sites for the shaft, the articulating wheel, the locking trigger, the central tubing (e.g., one or more conduits), and other accessory items to maintain a stable fit. The size and shape of the handle were carefully chosen to provide a comfortable fit to the user with many hand sizes. Also, keeping all user interface components in the midline of the device allows for accessibility with right or left-handed users.

Articulating Wheel and Knob (also referred to herein as a ratcheting mechanism and adjustment lever): A plastic wheel that has an extending knob for the user to push or pull with their thumb. When the user does this, the wheel can rotate within the handle casing and pull one of the drive wires in tension to produce tip articulation.

Locking Trigger: A spring-loaded trigger with teeth to interact with the wheel locking gear. This trigger is always engaged to prevent wheel rotation unless the trigger is squeezed by the user. The size and angle of the external portion of the trigger were designed carefully to fit ergonomically within the user's grip.

Articulating Wheel Locking Gear: A toothed gear that is attached to the articulating wheel. When engaged with the locking trigger, the wheel will not be able to rotate.

Central Tubing (also referred to herein as a one or more conduits): A plastic tube that runs from the most distal and inferior portion of the device handle, through the central axis of the shaft, and terminates at the distal face of the tip. This tubing allows for the advancement and containment of the optical fiber cable to the device tip for light delivery.

Spring Housing: A plastic housing built into the device handle to hold the springs to engage the locking trigger. The design allows the spring to be put under compression to allow constant locking capabilities and provide some resistance for the user to feel when the trigger is pulled.

Drive Wires (also referred to herein as guidewires): Metallic wires that connect the articulating tip to the articulating wheel. Pulling one of the wires in tension produces tip articulation in one direction.

Drive Wire Channels (also referred to herein as one or more conduits): Plastic extensions inside the device handle to channel the drive wires smoothly from the articulating wheel into the narrower diameter of the device shaft.

Trigger Pin: A steel bolt to provide a strong axis of rotation for the trigger mechanism when pulled by the user.

### Example 2: Photodynamic Therapy for Intra-Procedure Disinfection of Abdominal Infection

Disclosed in the following example is a novel photodynamic therapy (PDT) device and method for disinfection of the abdomen with PDT during surgical intervention for acute abdominal infection, particularly perforated appendicitis. In some embodiments, the disclosed PDT method comprises laparoscopic visualization of the infected region, delivery of the photosensitizing agent to the abdomen, and delivery of therapeutic illumination to the treatment region with an optical applicator.

In perforated appendicitis and other acute abdominal maladies, widespread infection can develop throughout the peritoneal cavity. This is typically treated with intravenous antibiotics, which results in extended hospital stays to allow time for antibiotics to control intra-abdominal infection due to stool and bacteria leakage. This effects tens of thousands of patients annually in the United States, particularly pediatric patients, with reported hospital stays ranging from 5-15 days. Shortening or eliminating the hospital stay could drastically improve patient experience and reduce hospital costs. PDT is a promising treatment modality for oncology and antimicrobial applications that relies on the excitation of light-sensitive drugs known as photosensitizers by visible light in order to generate reactive oxygen species. It was anticipated that PDT could be used to disinfect the peritoneal cavity or other intra-abdominal spaces during or immediately after surgical procedures such as appendectomy. As the antimicrobial effects of PDT occur on the timescale of hours, rather than the days required for traditional antibiotics, PDT can significantly reduce hospital stays for these patients. While PDT has been used to treat other infections, it has never been applied to intra-abdominal infection, which requires the novel approach described here.

In some embodiments, the disclosed PDT device and method comprises a means to deliver a photosensitive drug to the peritoneal cavity, a means to deliver light to the appropriate region during or following laparoscopic surgery, and the administration of PDT to the abdominal cavity. All components must therefore be compatible with a laparoscopic approach and not require additional surgical access.

In the initial conception, methylene blue was chosen as the photosensitizer, as preliminary results using this drug were displayed, as described herein. This drug could be delivered to the entire peritoneal cavity by filling the cavity with diluted methylene blue, which was performed in animal models. The photosensitizer could also be delivered locally by spraying the drug onto the surface of the peritoneal cavity with a device similar to a laparoscopic suction and irrigation system. This differs from oncology applications of PDT, where the photosensitive drug is delivered systemically by intravenous injection prior to the procedure. In the case of intraabdominal infection, it was hypothesized that the systemic approach would deliver insufficient photosensitizer to the bacteria for an efficacious outcome.

FIG. 1J is an image depicting a side view of an exemplary PDT device according to aspects of the present invention. Application to the peritoneal cavity also requires a novel approach to light delivery. Prior PDT applications have generally used surface illumination for regions of the body such as the skin, or the insertion of optical fibers directly into a mass for larger or deeper tumors. In the disclosed PDT method, a large region of the peritoneal cavity is illuminated through laparoscopic access. Therefore, a laparoscopic light delivery device (e.g., PDT device), with a similar form factor to commercially available cryoablation probes is disclosed herein. An exemplary drawing of this device is shown in FIG. 1J. In some embodiments, the PDT device comprises a flexible optical fiber with a flat-cleaved tip, enclosed in a controllable sleeve. The proximal end of this optical fiber would be connected to a laser or lamp source of the appropriate wavelength. The controllable sleeve would be steered by the surgeon, to allow directed illumination at the wall of the peritoneal cavity. As the laparoscopic procedure involves access for a camera, the illuminated region could be directly visualized in order to ensure that the entire infected region is treated. For particularly widespread infection, the cavity could additionally be filled with a scattering emulsion to homogenize the light dose at the cavity wall.

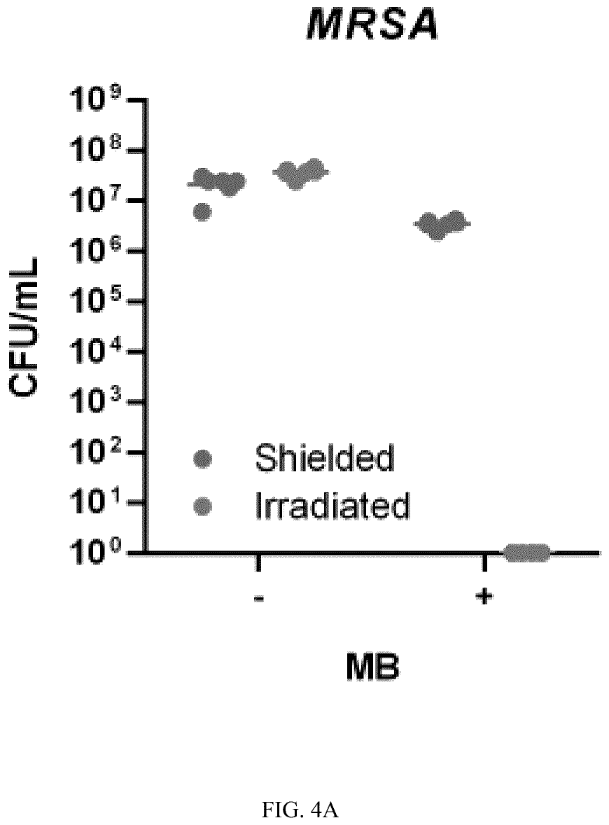

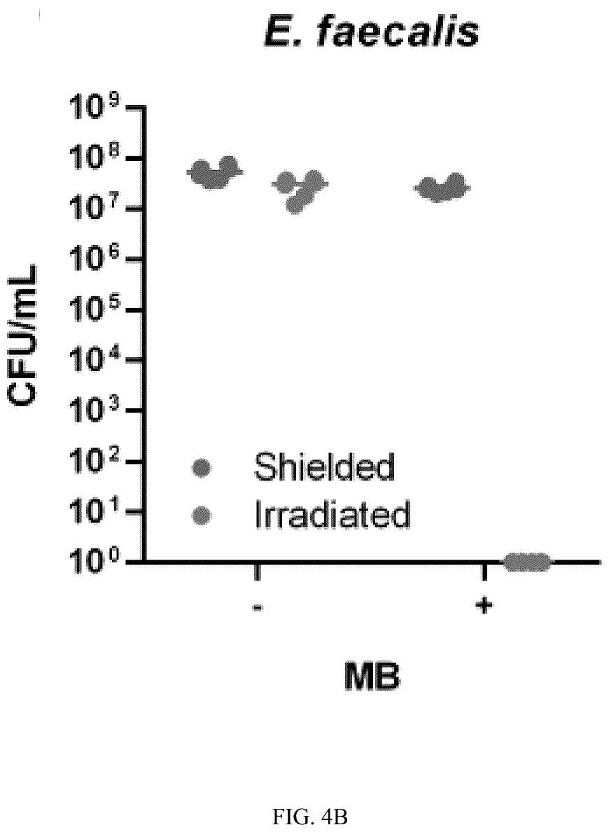

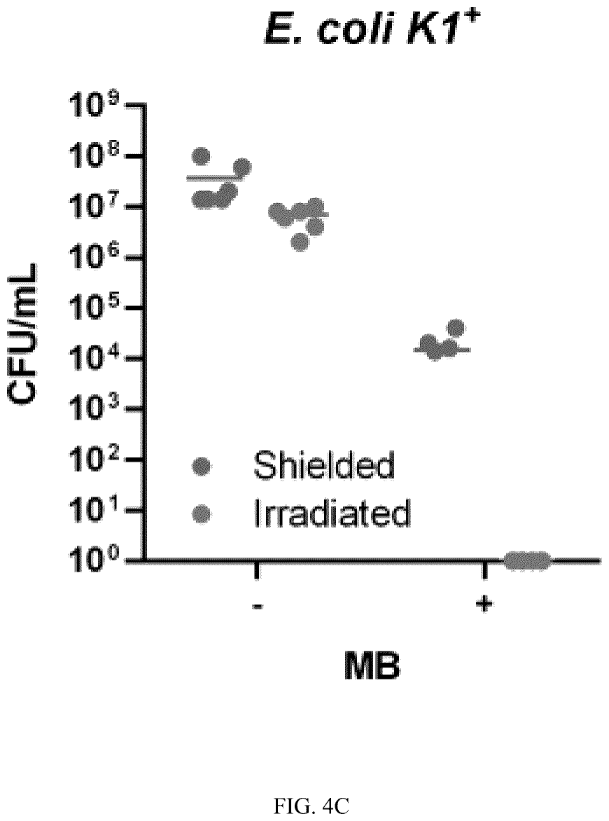

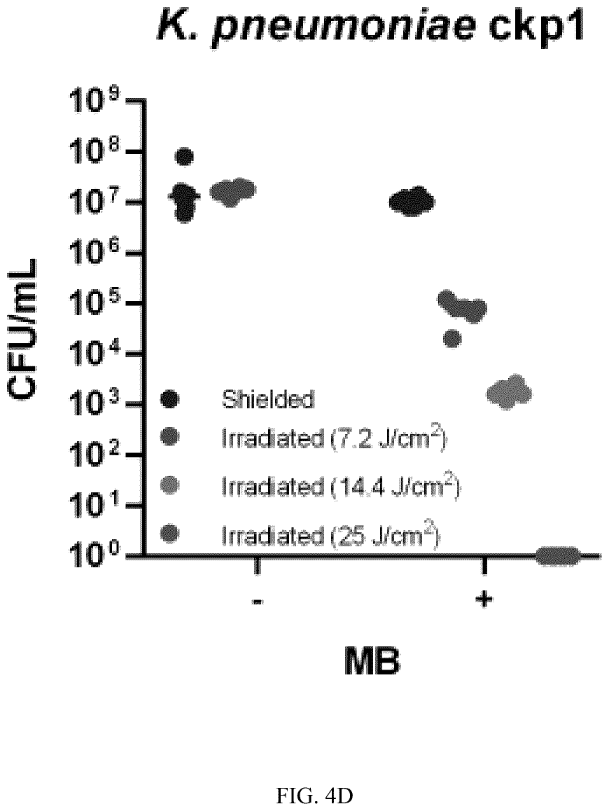

FIGS. 4A-4D are plots showing a reduction of colony forming units in four representative bacteria species following in vitro photodynamic therapy using the disclosed therapy device and method. FIG. 4A is a plot showing CFU/ml of Methicillin Resistant Staphylococcus Aureus (MRSA) after PDT treatment, with and without Methylene Blue (MB). FIG. 4B is a plot showing CFU/ml of Enterococcus Faecalis (E. Faecalis) after PDT treatment, with and without MB. FIG. 4C is a plot showing CFU/ml of Escherichia coli (E. coli) K1+after PDT treatment, with and without MB. FIG. 4D is a plot showing CFU/ml of Klebsiella pneumoniae ckp1 after PDT treatment, with and without MB.

PDT experiments in vitro were performed for four representative bacterial species that are commonly found in human abdominal experiments. As shown in FIGS. 4A-4D, using the disclosed PDT method resulted in a significant reduction in colony forming units (CFU) for all bacteria studied. This agrees with previously published in vitro data and motivates exploration of disinfection in a rabbit model. This will include surgical induction of perforated appendicitis, followed by PDT of the subsequent abdominal infection. Data collection in rabbits was performed.

The disclosed PDT device and method was also used for the treatment of deep tissue abscess cavities. No adverse or serious adverse events were encountered during this trial. These encouraging results motivate the current invention, which discloses a novel approach to drug and light delivery, as well as a laparoscopic visualization of the treatment region.

### Example 3: Photodynamic Therapy for Perforated Appendicitis

Appendicitis is the most common general surgical condition affecting children. The condition contributes to more morbidity & health care resource utilization than any other pediatric general surgical condition. Approximately ˜30% of pediatric cases are considered complicated (or perforated) appendicitis. In addition, this specific subpopulation suffers the majority of the morbidity. Of this subpopulation, 13% required drain placement after appendectomy, 11% return to the emergency department (ED) within 30 days, 34.5% required postoperative imaging, and overall have a mean length of stay: 6.48 (3.6) days.

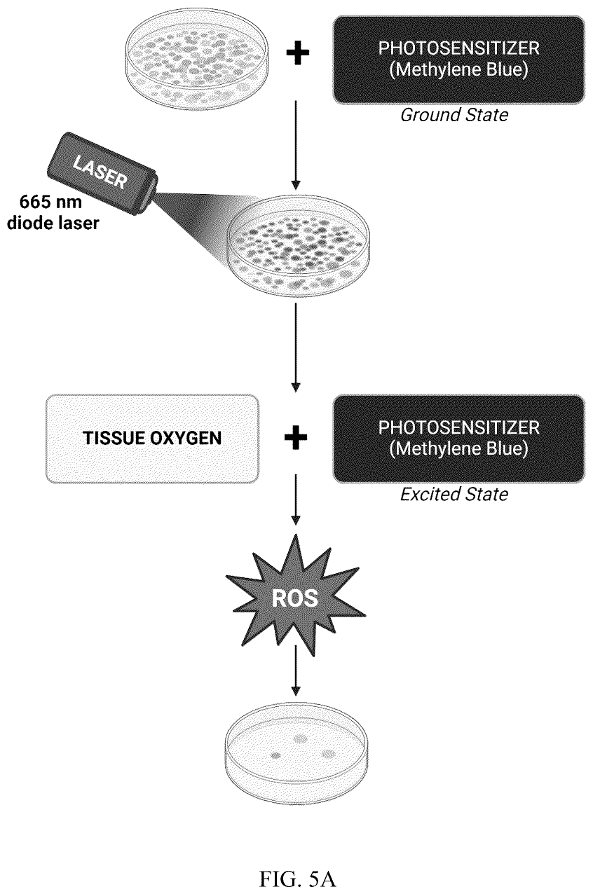

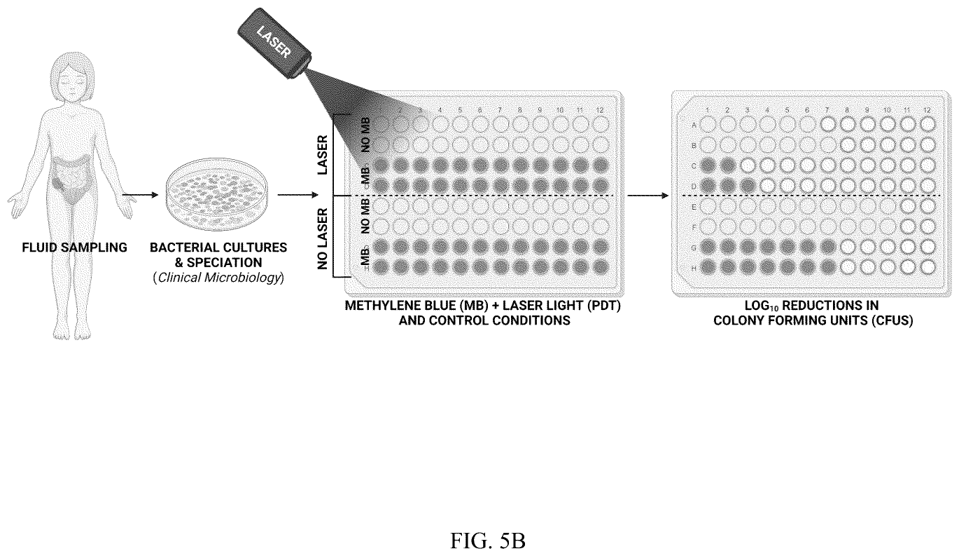

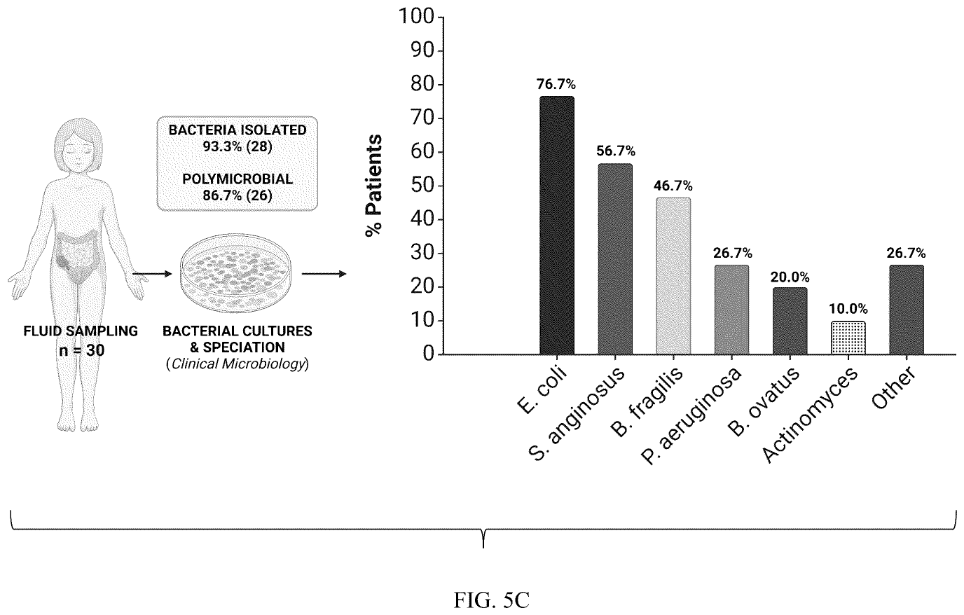

Disclosed herein are examples showing proof-of-concept for antimicrobial photodynamic therapy (PDT) as a treatment of intra-abdominal infection in complicated appendicitis. Demonstrated in these examples are the efficacy of the therapy against bacteria isolated from human samples, as well as a rabbit model of perforated appendicitis. FIG. 5A is a diagram depicting an exemplary PDT method according to aspects of the present invention. FIG. 5B is a diagram depicting an exemplary experimental setup for testing the disclosed PDT method.

PDT has been shown to have effective antimicrobial action and does not result in acquired bacterial resistance. Previous clinical success includes the treatment of endodontic infections, onychomycosis, chromoblastomycosis, and intra-abdominal abscess. It has been shown that methylene blue PDT (MB-PDT) is a suitable method for treating antibiotic-resistance biofilms on endotracheal (ET) tubes.

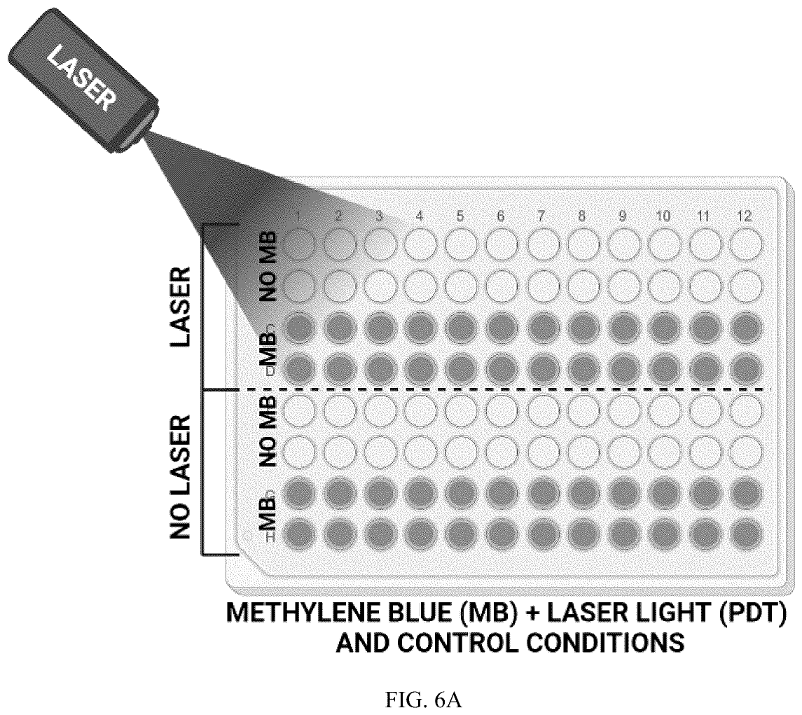

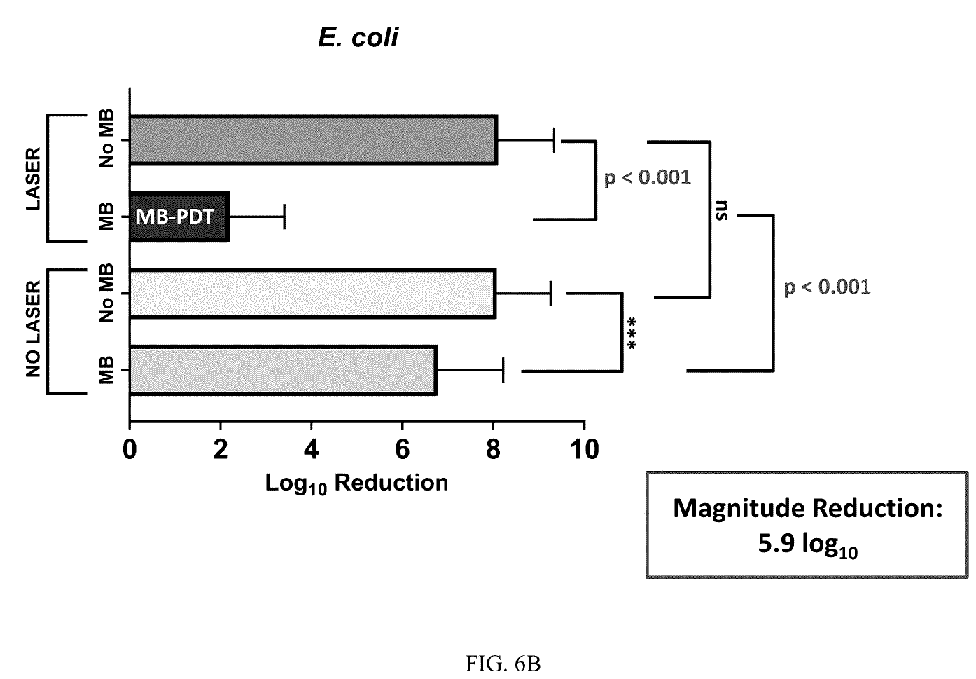

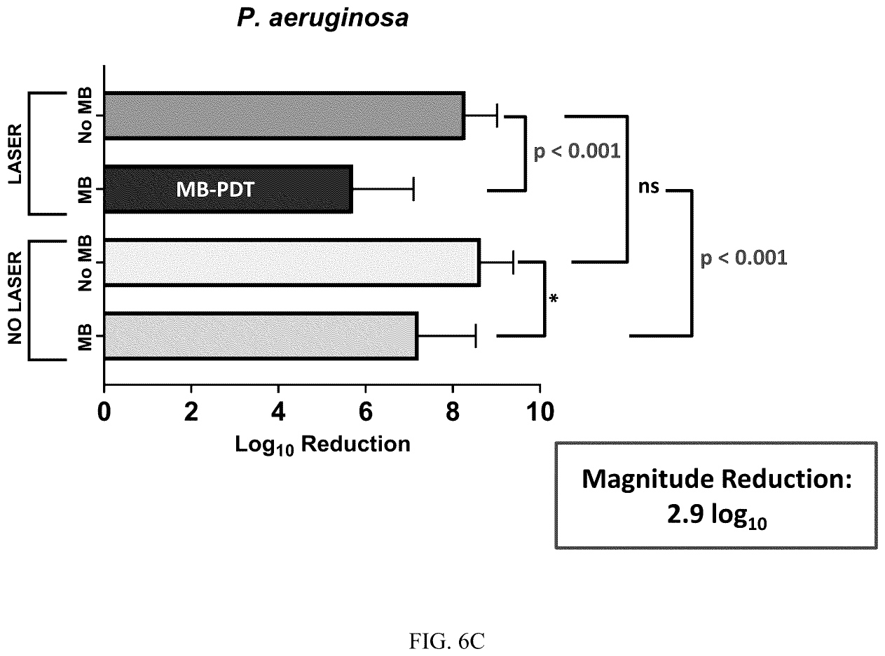

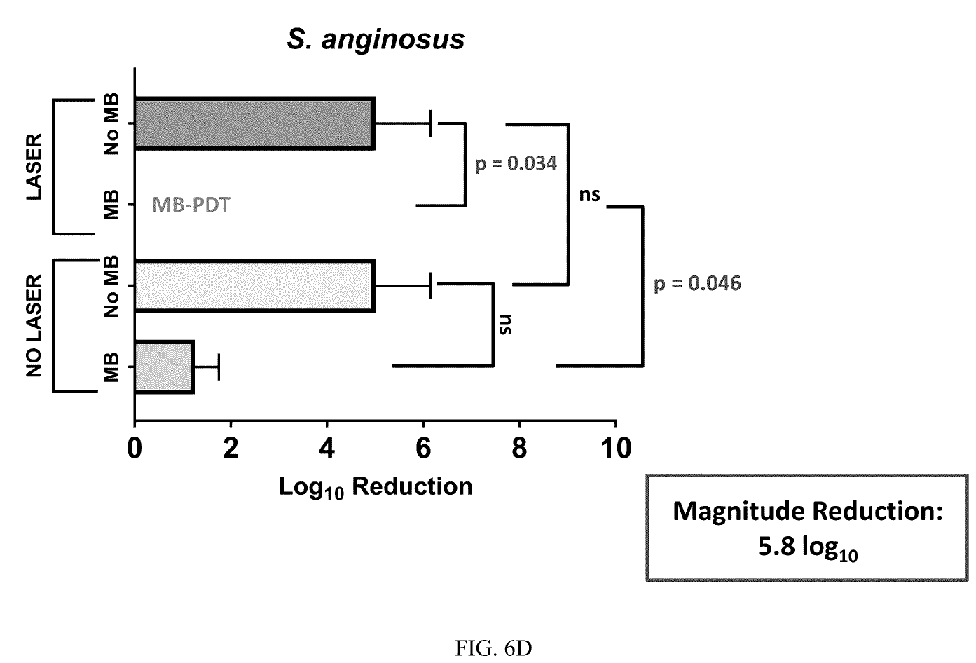

FIG. 5C is a diagram (left) showing the biological fluid sampling statistics and the clinical microbiology of the samples, and a plot (right) showing the percent speciation of the samples. FIG. 6A is a diagram depicting an enlarged view of the exemplary experimental setup of FIG. 5B showing a light source irradiating a well plate. FIG. 6B is a plot showing the results of the experiment of FIG. 5B showing a reduction of E. coli. FIG. 6C is a plot showing the results of the experiment of FIG. 5B showing a reduction of Pseudomonas aeruginosa. FIG. 6D is a plot showing the results of the experiment of FIG. 5B showing a reduction of Streptococcus anginosus.

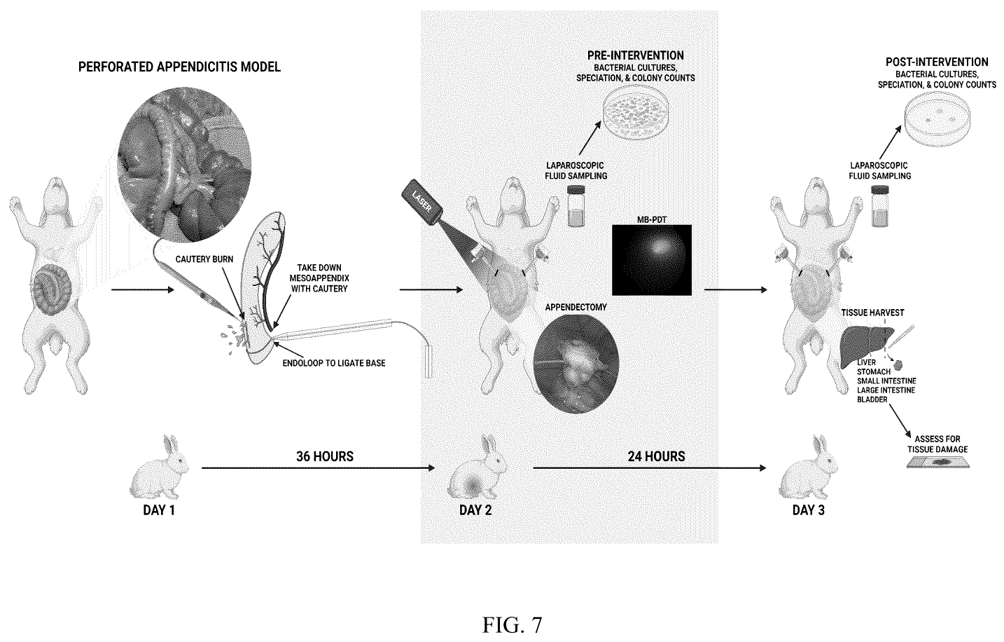

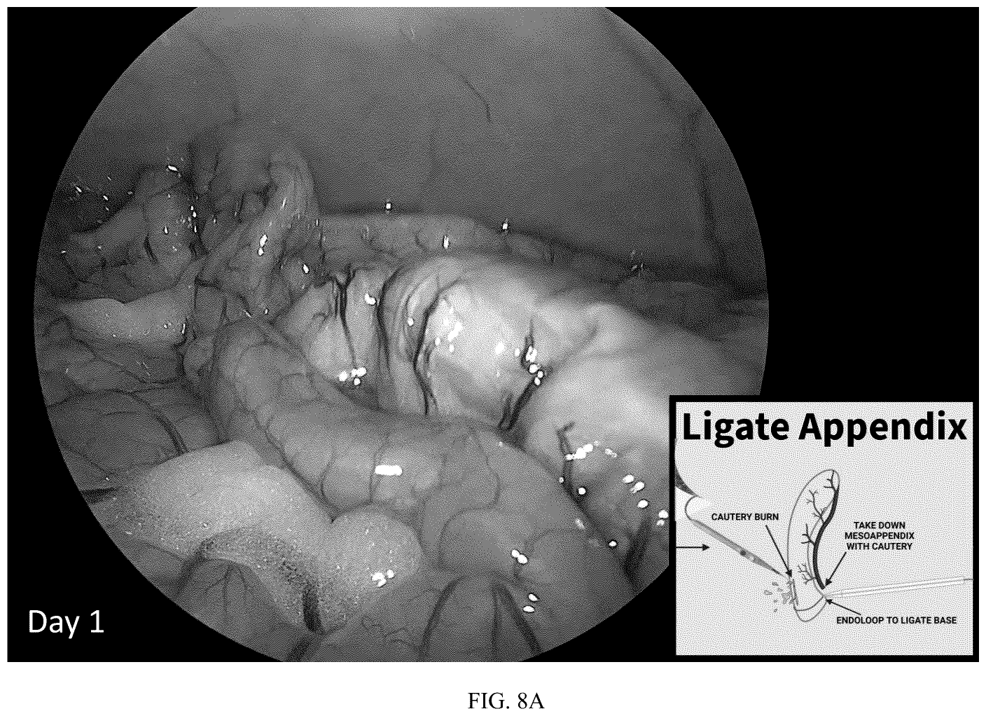

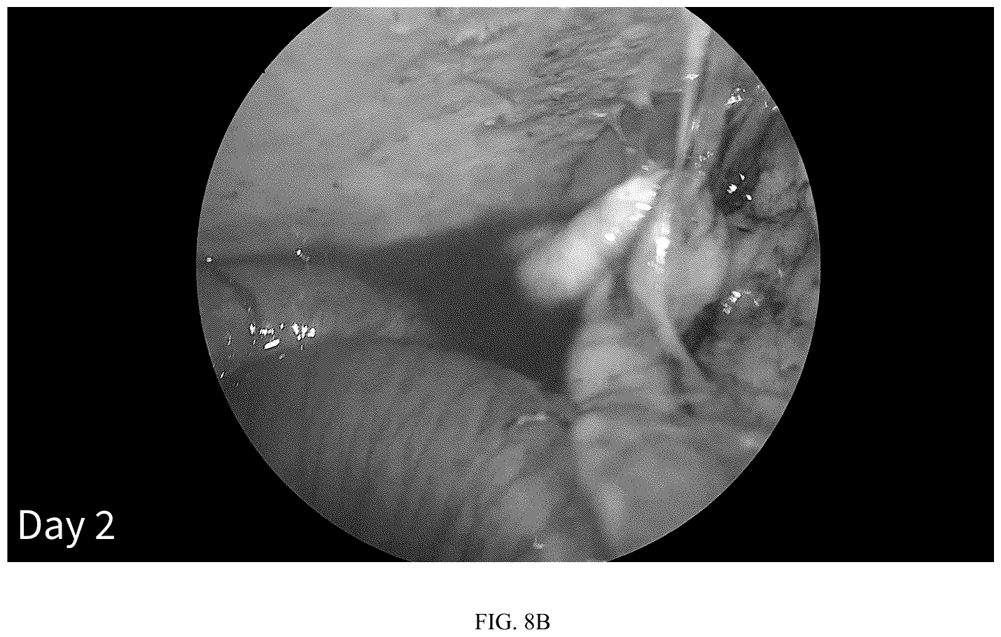

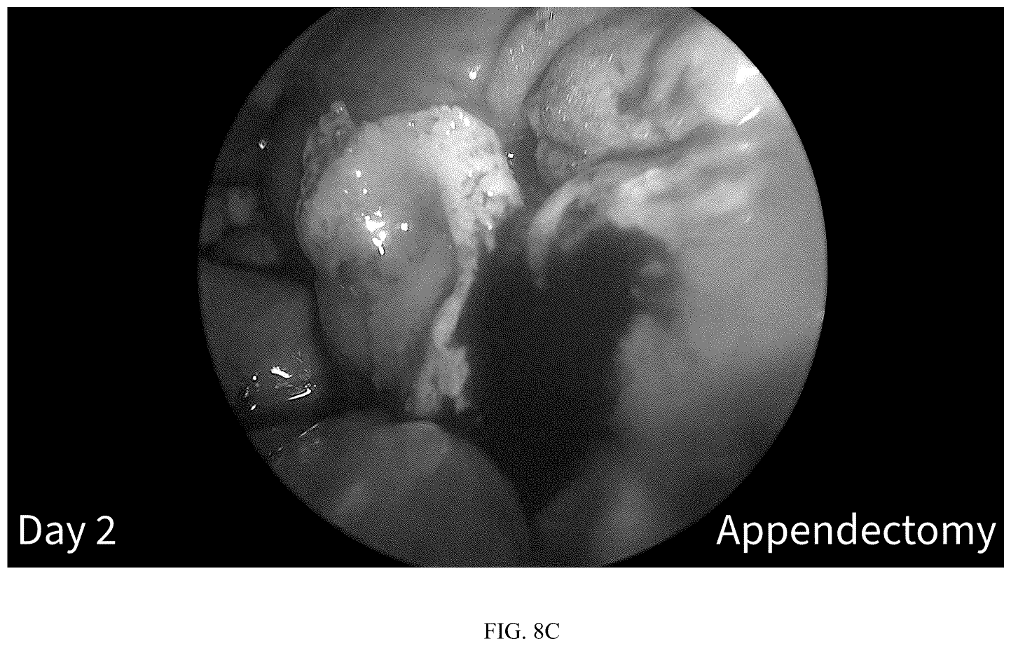

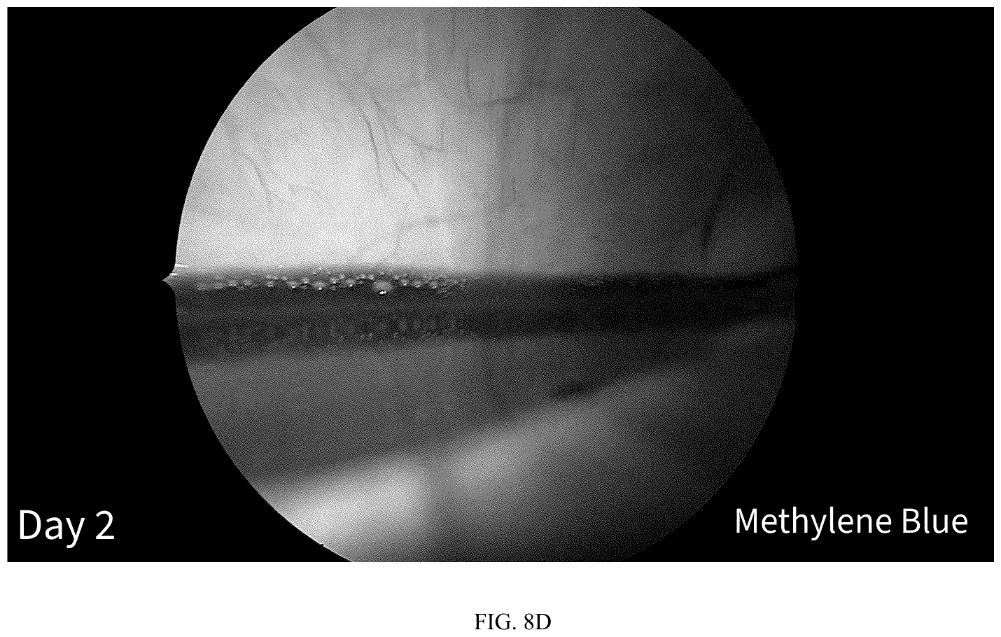

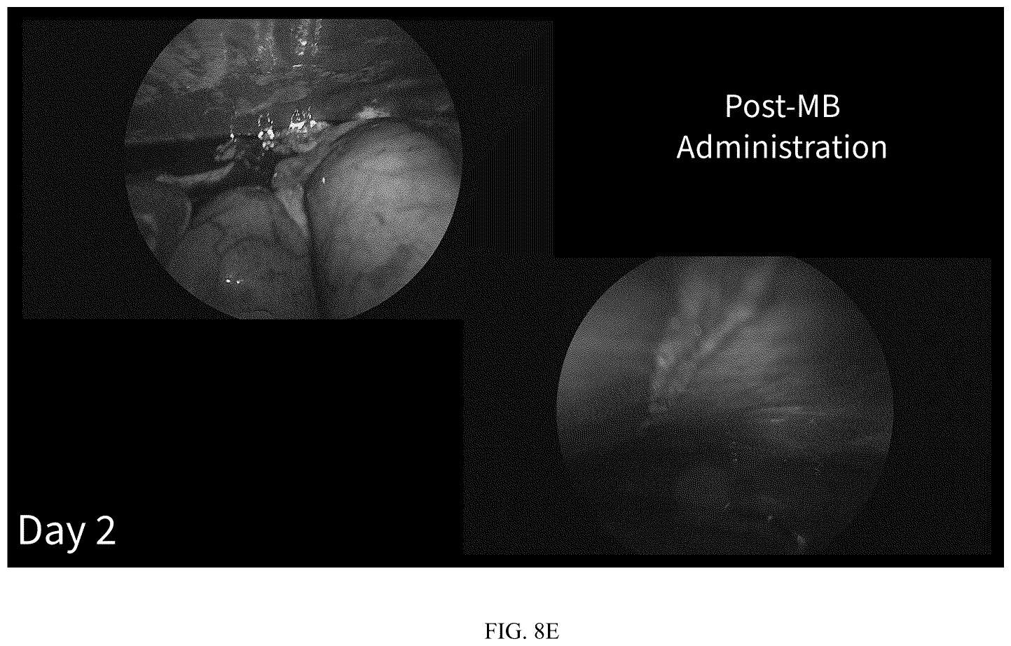

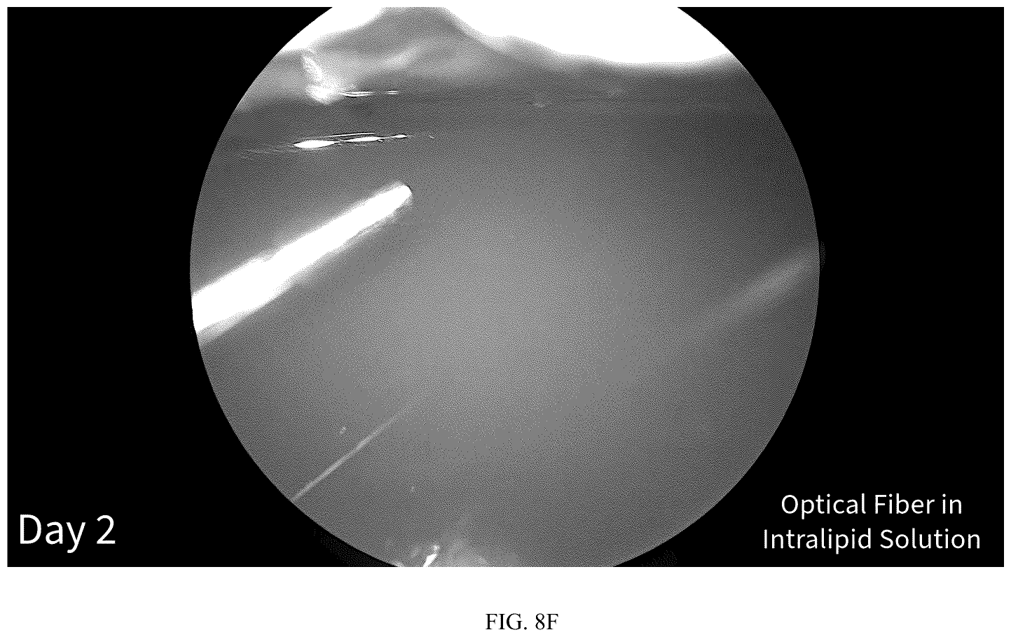

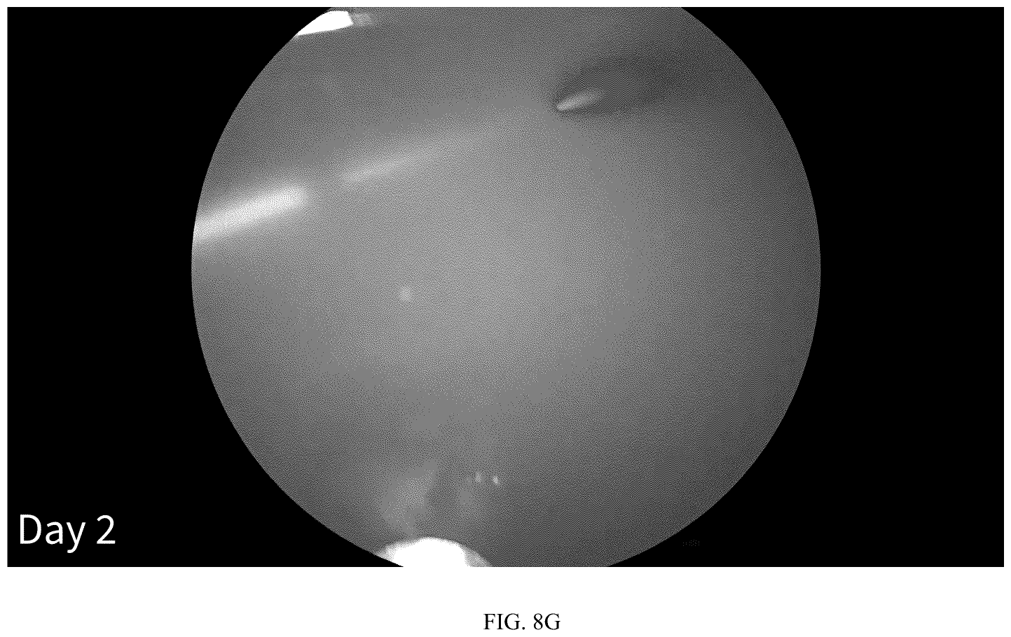

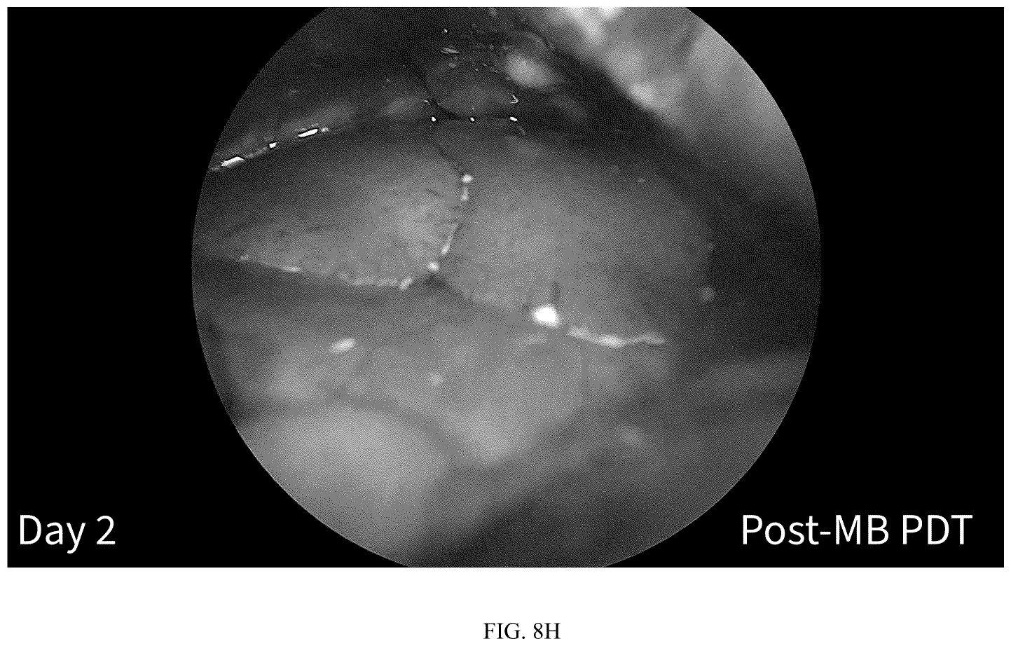

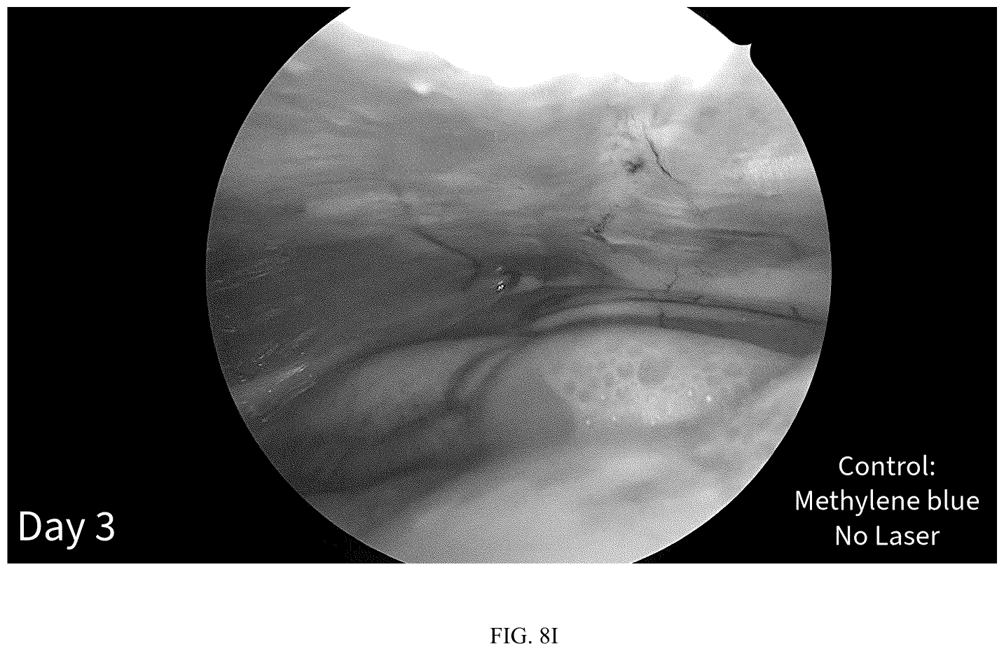

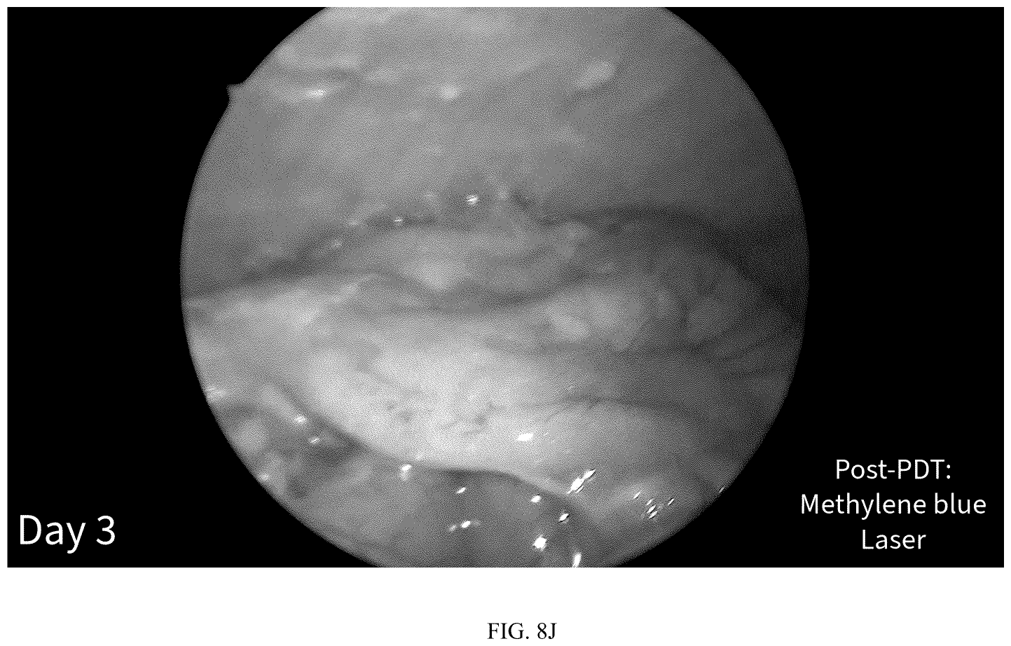

FIG. 7 is a diagram depicting an exemplary perforated appendicitis model utilizing the disclosed PDT method. FIGS. 8A-8J are a series of images showing the steps of the perforated appendicitis model of FIG. 7, as performed on a subject. FIG. 8A is an image showing an appendix on day 1 before an appendectomy. FIG. 8B is an image showing the appendix prior to appendectomy on day 2. FIG. 8C is an image showing the appendiceal stump after appendectomy on day 2. FIG. 8D is an image showing the administration of MB on day 2. FIG. 8E is an image showing post-MB administration and administration of intralipid for light scattering on day 2. FIG. 8F is an image showing the optical fiber of the disclosed PDT device in intralipid solution on day 2. FIG. 8G is an image showing illumination of the optical fiber on day 2. FIG. 8H is an image showing the abdomen post-MB PDT after the appendectomy on day 3. A control subject was also used that did not receive irradiation with a light source. FIG. 8I is an image showing the results of the control subject that was treated with only MB. FIG. 8J shows the results of the subject that received MB and was irradiated with a light source.

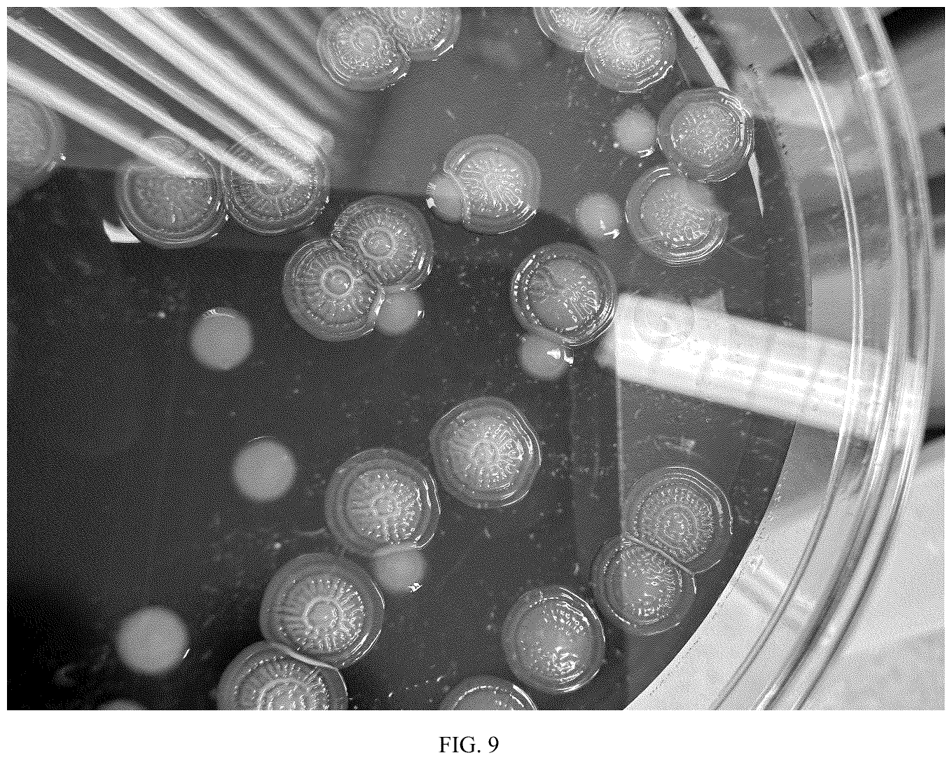

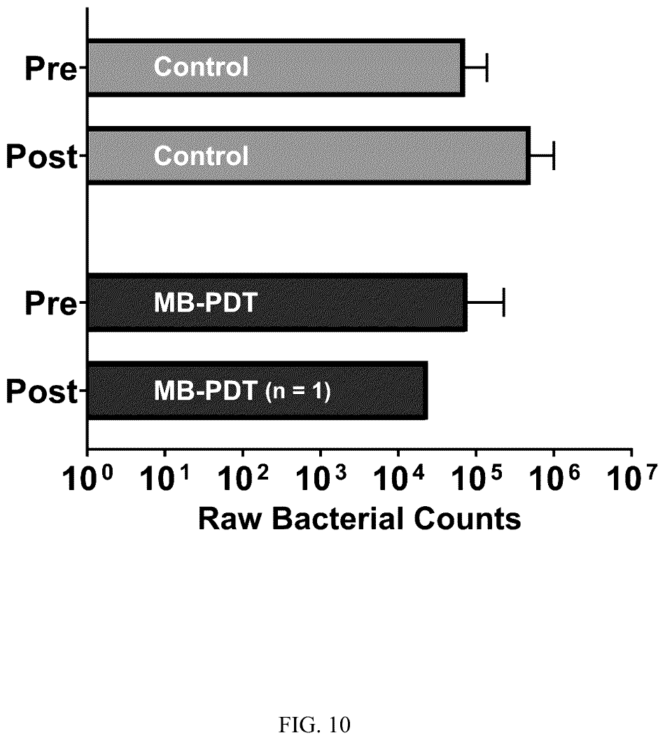

FIG. 9 is an image of a culture of fluid sampled from the subject shown in FIGS. 8A-8J showing a reliable initiation of perforated appendicitis including clinical evidence of peritonitis and polymicrobial infection. FIG. 10 is a plot showing the results for the pre- and post-fluid sampling of the subject shown in FIGS. 8A-8J. The results display a successful deployment of MB-PDT.

In conclusion, MB-PDT effectively kills bacteria from human subjects with perforated appendicitis in vitro. The magnitude of kill depends on bacterial species and colony. Disclosed in this example was a successful creation of a rabbit model of perforated appendicitis with successful laparoscopic deployment of MB-PDT in rabbit model with encouraging results.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A photodynamic therapy (PDT) device, comprising:
a cannula having proximal and distal ends and a lumen therebetween;
a flexible tube having proximal and distal ends, wherein the proximal end is attached to and extending out from the distal end of the cannula;
a handle portion attached to the proximal end of the cannula comprising a housing with an adjustment mechanism, wherein adjusting the adjustment mechanism articulates the flexible tube;
one or more conduits extending from at least the handle portion through the cannula to the distal end of the flexible tube; and
a light source positioned at the distal end of the flexible tube, configured to irradiate in a distal direction.

2. The device of claim 1, further comprising first and second guidewires positioned inside first and second conduits of the one or more conduits, each guide wire connected to the adjustment mechanism and the flexible tube.

3. The device of claim 2, wherein the light source comprises a fiberoptic cable extending through a third conduit of the one or more conduits, wherein the third conduit extends out of the handle portion proximally.

4. The device of claim 3, wherein the adjustment mechanism comprises an adjustment lever and ratcheting mechanism, each at least partially housed inside the handle portion, wherein the ratcheting mechanism is connected to the first and second guidewires and rotated by the adjustment lever.

5. The device of claim 4, wherein the adjustment mechanism comprises a locking lever extending out from the handle portion connected to the ratcheting mechanism, and wherein the adjustment mechanism articulates the flexible tube along at least one axis, and the locking lever locks the position of the adjustment mechanism.

6. The device of claim 5, wherein the adjustment mechanism articulates the flexible tube within a range, wherein the range is between 0°-270°.

7. The device of claim 6, further comprising a fourth conduit of the one or more conduits configured to dispense one or more fluids, wherein the one or more fluids comprise one or more agents selected from the group consisting of: therapeutic agent, photosensitizing agent, photosensitive therapeutic agent, antimicrobial agent, antimicrobial photosensitive therapeutic agent, and antibiotic agent.

8. The device of claim 7, wherein the photosensitizing agent comprises at least one selected from the group consisting of: methylene blue, indocyanine green, and 5-Aminolevulinic acid.

9. The device of claim 7, wherein the one or more fluids further comprise a light scattering emulsion selected from: lipid emulsion, sterile lipid emulsion, intravenous fat emulsion, intralipid, and nutrilipid.

10. The device of claim 7, further comprising a fifth conduit of the one or more conduits comprising one or more sensors, wherein the one or more sensors are selected from the group consisting of: detector, photodiode, thermistor, transducer, photodiode configured to detect fluorescence, spectrometer, and fiber-coupled spectrometer.

11. The device of claim 10, further comprising a camera positioned at the distal end of the flexible tube.

12. The device of claim 10, further comprising a sixth conduit of the one or more conduits configured to produce suction to remove fluid dispensed from the fourth conduit, or to retrieve a biological sample, wherein the biological sample comprises any of: fluid, biological fluid, purulent fluid, tissue, excised tissue, microbe, and microbial community.

13. The device of claim 1, wherein the light source is configured to emit one or more wavelengths of light ranging between 500 nm-900 nm, between 600 nm-850 nm, between 625 nm-785 nm, between 615 nm-645 nm, between 650 nm-680 nm, between 770 nm and 800 nm, or optionally configured to emit one or more target wavelengths, wherein the wavelengths are selected from: 633 nm, 665 nm, and 785 nm.

14. A method for performing PDT comprising:
providing a PDT device comprising:
a cannula having proximal and distal ends and a lumen therebetween;
a flexible tube having proximal and distal ends, wherein the proximal end is attached to and extending out from the distal end of the cannula;
a handle portion attached to the proximal end of the cannula comprising a housing with an adjustment mechanism, wherein adjusting the adjustment mechanism articulates the flexible tube;
one or more conduits extending from at least the handle portion through the cannula to the distal end of the flexible tube; and
a light source positioned at the distal end of the flexible tube, configured to irradiate in a distal direction;
delivering one or more fluids to an area of interest of a subject;
positioning the distal end of the flexible tube near the area of interest;
adjusting the distal end of the flexible tube to the area of interest; and
irradiating at least a portion of the area of interest with the light source.

15. The method of claim 14, wherein the one or more fluids are delivered to the area of interest through the one or more conduits, and wherein the one or more fluids comprise one or more agents selected from the group consisting of: therapeutic agent, photosensitizing agent, photosensitive therapeutic agent, antimicrobial agent, antimicrobial photosensitive therapeutic agent, and antibiotic agent.

16. The method of claim 15, wherein the photosensitizing agent comprises at least one selected from the group consisting of: methylene blue, indocyanine green, and 5-Aminolevulinic acid.

17. The method of claim 15, wherein the one or more fluids further comprise a light scattering emulsion selected from: lipid emulsion, sterile lipid emulsion, intravenous fat emulsion, intralipid, and nutrilipid.

18. The method of claim 14, wherein the light source is configured to emit one or more wavelengths of light ranging between 500 nm-900 nm, between 600 nm-850 nm, between 625 nm-785 nm, between 615 nm-645 nm, between 650 nm-680 nm, between 770 nm and 800 nm, or optionally configured to emit one or more target wavelengths, wherein the wavelengths are selected from: 633 nm, 665 nm, and 785 nm.

19. The method of claim 14, further comprising the step of removing at least a portion of the one or more fluids through the one or more conduits.

20. The method of claim 14, further comprising retrieving a biological sample from the area of interest of the subject, wherein the biological sample is retrieved through the one or more conduits.

21. The method of claim 14, further comprising imaging the area of interest with a camera positioned at the distal end of the flexible tube.

22. The method of claim 14, further comprising measuring fluorescence at the area of interest with a sensor positioned at the distal end of the flexible tube.

23. The method of claim 14, wherein the area of interest of the subject is selected from the group consisting of: bodily cavity, abdominal cavity, peritoneum, chest cavity, gastrointestinal tract, thoracic cavity, spinal cavity, pelvic cavity, abdominopelvic cavity, inside the bladder, inside the tracheobronchial tree, spinal canal, inside the GI tract, inside the mouth, inside the pharynx.
